(19) United States
(12) Patent Application Publication
(10) Pub. No.: US 2007/0027634 A1
(43) Pub. Date: Feb. 1, 2007

(54) Molecular hepatotoxicology modeling

(76) Inventors: Donna L. Mendrick, Gaithersburg, MD (US); Mark W. Porter, Gaithersburg, MD (US); Kory R. Johnson, Gaithersburg, MD (US); Brandon Higgs, Gaithersburg, MD (US); Arthur L. Castle, Gaithersburg, MD (US); Michael Elashoff, Gaithersburg, MD (US)

Correspondence Address: MORGAN LEWIS & BOCKIUS LLP, 1111 PENNSYLVANIA AVENUE NW, WASHINGTON, DC, 20004, US

(21) Appl. No.: 10/501,933

(22) PCT Filed: Jan. 31, 2003
(86) PCT No.: PCT/US03/03194

§ 371 Date: Oct. 27, 2004

Related U.S. Application Data
(60) Provisional application No. 60/364,055, filed on Mar. 15, 2002.
(60) Provisional application No. 60/364,045, filed on Mar. 15, 2002.
(60) Provisional application No. 60/436,643, filed on Dec. 30, 2002.

(30) Foreign Application Priority Data
Jan. 31, 2002 (US) 10/060087

(51) Int. Cl. C12Q 1/68; G06F 19/00; G01N 33/48; G01N 33/50

(52) U.S. Cl. 702/20; 435/6

(57) ABSTRACT

The present invention is based on the elucidation of the global changes in gene expression and the identification of toxicity markers in liver tissues or cells exposed to a known toxin. The genes may be used as toxicity markers in drug screening and toxicity assays. The invention includes a database of genes characterized by liver toxin-induced differential expression that is designed for use with microarrays and other solid-phase probes.

### RELATED APPLICATIONS

This application claims priority to U.S. Provisional Application 60/364,045 filed on Mar. 15, 2002, 60/364,055 filed on Mar. 15, 2002, and 60/436,643 filed on Dec. 30, 2002, and is a continuation-in-part of pending U.S. application Ser. No. 10/060,087 filed Jan. 31, 2002. In addition, this application is related to U.S. Provisional Application 60/222,040, 60/244,880, 60/290,029, 60/290,645, 60/292,336, 60/295,798, 60/297,457, 60/298,884, 60/303,459, and 60/331,273, as well as to pending U.S. application Ser. No. 09/917,800, filed Jul. 31, 2001, all of which are herein incorporated by reference in their entirety.

### SEQUENCE LISTING SUBMISSION ON COMPACT DISC

The Sequence Listing submitted concurrently herewith on compact disc is herein incorporated by reference in its entirety. Four copies of the Sequence Listing, one on each of four compact discs are provided. Copies 1, 2, and 3 are identical. Copies 1, 2, and 3 are also identical to the CRF. Each electronic copy of the Sequence Listing was created on Jan. 30, 2003 with a file size of 5795 KB. The file names are as follows: Copy 1-g15038us01.txt; Copy 2-g15038us01.txt; Copy 3-g15038us01.txt; and CRF-g15038us01.txt.

### BACKGROUND OF THE INVENTION

The need for methods of assessing the toxic impact of a compound, pharmaceutical agent or environmental pollutant on a cell or living organism has led to the development of procedures which utilize living organisms as biological monitors. The simplest and most convenient of these systems utilize unicellular microorganisms such as yeast and bacteria, since they are most easily maintained and manipulated. Unicellular screening systems also often use easily detectable changes in phenotype to monitor the effect of test compounds on the cell. Unicellular organisms, however, are inadequate models for estimating the potential effects of many compounds on complex multicellular animals, as they do not have the ability to carry out biotransformations to the extent or at levels found in higher organisms.

The biotransformation of chemical compounds by multicellular organisms is a significant factor in determining the overall toxicity of agents to which they are exposed. Accordingly, multicellular screening systems may be preferred or required to detect the toxic effects of compounds. The use of multicellular organisms as toxicology screening tools has been significantly hampered, however, by the lack of convenient screening mechanisms or endpoints, such as those available in yeast or bacterial systems. In addition, previous attempts to produce toxicology prediction systems have failed to provide the necessary modeling data and statistical information to accurately predict toxic responses (e.g., WO 00/12760, WO 00/47761, WO 00/63435, WO 01/32928 and WO 01/38579).

### SUMMARY OF THE INVENTION

The present invention is based on the elucidation of the global changes in gene expression in tissues or cells exposed to known toxins, in particular hepatotoxins, as compared to unexposed tissues or cells as well as the identification of individual genes that are differentially expressed upon toxin exposure.

In various aspects, the invention includes methods of predicting at least one toxic effect of a compound, predicting the progression of a toxic effect of a compound, and predicting the hepatoxicity of a compound. The invention also includes methods of identifying agents that modulate the onset or progression of a toxic response. Also provided are methods of predicting the cellular pathways that a compound modulates in a cell. The invention includes methods of identifying agents that modulate protein activities.

In a further aspect, the invention provides probes comprising sequences that specifically hybridize to genes in Tables 1-5WWW. Also provided are solid supports comprising at least two of the previously mentioned probes. The invention also includes a computer system that has a database containing information identifying the expression level in a tissue or cell sample exposed to a hepatotoxin of a set of genes comprising at least two genes in Tables 1-5WWW.

The invention further provides a core set of genes in Tables 5A-5WWW from which probes can be made and attached to solid supports. These core genes serve as a preferred set of markers of liver toxicity and can be used with the methods of the invention to predict or monitor a toxic effect of a compound or to modulate the onset or progression of a toxic response.

### DETAILED DESCRIPTION

Many biological functions are accomplished by altering the expression of various genes through transcriptional (e.g. through control of initiation, provision of RNA precursors, RNA processing, etc.) and/or translational control. For example, fundamental biological processes such as cell cycle, cell differentiation and cell death are often characterized by the variations in the expression levels of groups of genes.

Changes in gene expression are also associated with the effects of various chemicals, drugs, toxins, pharmaceutical agents and pollutants on an organism or cells. For example, the lack of sufficient expression of functional tumor suppressor genes and/or the over expression of oncogene/protooncogenes after exposure to an agent could lead to tumorgenesis or hyperplastic growth of cells (Marshall (1991) Cell 64: 313-326; Weinberg (1991) Science 254:1138-1146). Thus, changes in the expression levels of particular genes (e.g. oncogenes or tumor suppressors) may serve as signposts for the presence and progression of toxicity or other cellular responses to exposure to a particular compound.

Monitoring changes in gene expression may also provide certain advantages during drug screening and development. Often drugs are screened for the ability to interact with a major target without regard to other effects the drugs have on cells. These cellular effects may cause toxicity in the whole animal, which prevents the development and clinical use of the potential drug.

The present inventors have examined tissue from animals exposed to the known hepatotoxins which induce detrimental liver effects, to identify global changes in gene expression induced by these compounds. These global changes in gene expression, which can be detected by the production of gene expression profiles, provide useful toxicity markers that can be used to monitor toxicity and/or toxicity progression by a test compound. Some of these markers may also be used to monitor or detect various disease or physiological states, disease progression, drug efficacy and drug metabolism.

Identification of Toxicity Markers

To evaluate and identify gene expression changes that are predictive of toxicity, studies using selected compounds with well characterized toxicity have been conducted by the present inventors to catalogue altered gene expression during exposure in vivo. In the present study, acetominophen, 2-acetylaminofluorene (2-AAF), acyclovir, ANIT, AY-25329, BI liver toxin, chloroform, bicalutamide, carbon tetrachloride, chloroform, CI-1000, clofibrate, colchicine, CPA, diclofenac, diflunisal, dimethylnitrosamine (DMN), dioxin, 17α-ethinylestradiol, gemfibrozil, hydrazine, indomethacin, LPS, menadione, phenobarbital, tacrine, thioacetamide, valproate, Wy-14643, and zileuton were selected as known hepatotoxins.

Aromatic and aliphatic isothiocyanates are commonly used soil fumigants and pesticides (Shaaya et al. (1995) Pesticide Science 44(3):249-253; Cairns et al. (1988) J Assoc Official Analytical Chemists 71(3):547-550). These compounds are also environmental hazards, because they remain as toxic residues in plants (Cemy et al. (1996) J Agricultural and Food Chemistry 44(12):3835-3839) and because they are released from the soil into the surrounding air (Gan et al. (1998) J Agricutural and Food Chemistry 46(3):986-990).

Exposure to α-naphthylisothiocyanate (ANIT) has been shown to increase serum levels of total bilirubin, alkaline phosphatase, serum glutamic oxaloacetic transaminase and serum glutamic pyruvic transaminase, while total bile flow was reduced, all of which are indications of severe biliary dysfunction. ANIT also induces jaundice and cholestatis (the condition caused by failure to secrete bile, resulting in plasma accumulation of bile substances, liver cell necrosis and bile duct obstruction) (Tanaka et al. (1993) Clinical and Experimental Pharmacology and Physiology 20:543-547). ANIT fails to produce extensive necrosis, but was found to produce inflammation and edema in the portal tract of the liver (Maziasa et al. (1991) Toxicol Appl Pharmacol 110:365-373). ANIT-induced hepatotoxicity may also characterized by cholangiolitic hepatitis and bile duct damage. Acute hepatotoxicity caused by ANIT in rats is manifested as neutrophil-dependent necrosis of bile duct epithelial cells (BDECs) and hepatic parenchymal cells. These changes mirror the cholangiolitic hepatitis found in humans (Hill (1999) Toxicol Sci 47:118-125).

Histological changes include an infiltration of polymorphonuclear neutrophils and elevated number of apoptotic hepatocytes (Calvo et al. (2001) J Cell Biochem 80(4):461-470). Other known hepatotoxic effects of exposure to ANIT include a damaged antioxidant defense system, decreased activities of superoxide dismutase and catalase (Ohta et al. (1999) Toxicology 139(3):265-275), and the release of proteases from the infiltrated neutrophils, alanine aminotransferase, cathepsin G, elastase, which mediate hepatocyte killing (Hill et al. (1998) Toxicol Appl Pharmacol 148(1):169-175).

The effects of the model compound 2-acetylaminofluorene (2-AAF), a strong carcinogen and liver tumor inducer, have been studied in rat livers. 2-AAF has been shown to cause changes in the mitochondria which trigger apoptosis and regenerative cell proliferation. These in turn, cause cirrhosis-like changes in the liver. Exposure to 2-AAF also produces elevated levels of ALT and AST, hemoglobin adducts and foci containing the placental form of glutathione S-transferase. Chromosome aberrations, micronuclei and sister-chromatid exchanges have also been observed (Bitsch et al. (2000) Toxicol Sci 55(1):44-51; Lorenzini et al. (1996) Carcinogenesis 17:1323-1329; Sawada et al. (1991) Mutat Res 251(1):59-69).

Acyclovir (9-[(2-hydroxyethyl)methyl]guanine, Zovirax®), an anti-viral guanosine analogue, is used to treat herpes simplex virus (HSV), varicella zoster virus (VZV) and Epstein-Barr virus (EBV) infections. The most common adverse effect of acyclovir treatment is damage to various parts of the kidney, particularly the renal tubules, although the drug can also cause damage to the liver and nervous system. Crystalluria, or the precipitation of crystals of acyclovir in the lumina of the renal tubules can occur (Fogazzi (1996) Nephrol Dial Transplant 11(2):379-387). If the drug crystallizes in the renal collecting tubules, obstructive nephropathy and tubular necrosis can result (Richardson (2000) Vet Hum Toxicol 42(6):370-371). Examination of biopsy tissues from affected patients showed dilation of the proximal and distal renal tubules, with loss of the brush border, flattening of the lining cells and focal nuclear loss (Becker et al. (1993) Am J Kidney Dis 22(4):611-615).

Liver damage in patients taking acyclovir is indicated clinically by abnormal liver function tests (http://www.hopkins-aids.edu/publications/book/ch6_acyclovir.html). Adverse effects in the liver include hepatitis, hyperbilirubinemia and jaundice (Physicians' Desk Reference, 56^{th} ed., p. 1707, Medical Economics Co. Inc., Montvale, N.J., 2002), although findings of hepatotoxicity in animals have not yet been published. Studies by the present inventors on rats treated with acyclovir have found elevated serum levels of BUN and creatinine. Decreased levels of ALT, AST and triglycerides (indicators of liver function) have also been found, but these may be attributed to kidney damage as well as to liver damage. While classic signs of hepatotoxicity in rats due to acyclovir administration have not been published, gene expression changes can be used to predict that the drug will be a liver toxin in humans.

Acetominophen (APAP) is a widely used analgesic and antipyretic agent that is an effective substitute for aspirin. Although acetaminophen does not have anti-inflammatory properties, it is preferably given to patients with ulcers or patients in whom prolonged clotting times would not be desirable. It also preferably taken by people who do not tolerate aspirin well.

Acetominophen is metabolized to N-acetyl-p-benzoquinoneimine (NAPQI) by N-hydroxylation in a cytochrome P450-mediated process. This highly reactive intermediate, which reacts with sulfhydryl groups in glutathione, and in other liver proteins following the depletion of glutathione, can cause centrilobular hepatic necrosis (particularly in zone 3), renal tubular necrosis, and hepatic and renal failure (Goodman and Gilman's The Pharmacological Basis of Therapeutics, Ninth Ed., Hardman et al., eds., pp. 631-633, McGraw-Hill, New York, 1996; Chanda et al. (1995) Hepatology 21(2):477-486). Less serious side effects include skin rashes (erythemas and urticarias) and allergic reactions.

Upon treatment of rats with acetaminophen, hepatotoxicity can be observed 24 hours after dosing, as determined by statistically significant elevations of ALT and AST in the serum and by hepatocellular necrosis visualized at the light microscopic level (Hessel et al. (1996) Braz J Med Biol Res 29(6):793-796; Bruck et al. (1999) Dig Dis Sci 44(6):1228-1235). High, but non-lethal, doses of acetaminophen given to rats also produced elevated levels of genes involved in hepatic acute phase response and liver cell maintenance and repair: arginase, beta-fibrinogen, alpha 1-acid glycoprotein, alpha-tubulin, histone 3, TGF beta and cyclin d. Expression levels of genes regulated by the cell cycle were decreased (Tygstrup et al. (1996) J Hepatol 25(2):183-190; Tygstrup et al. (1997) J Hepatol 27(1):156-162). In mice, expression levels of genes that encode growth arrest and cell cycle regulatory proteins were increased, along with expression levels of stress-induced genes, transcription factor LRG-21, SOCS-2 (cytokine signaling repressor) and PAI-1 (plasminogen activator inhibitor-1) (Reilly et al. (2001) Biochem Biophys Res Comm 282(1):321-328).

AY-25329, a proprietary compound, is a phenothiazine that has been shown to be toxic in liver and in kidney tissue, where it can cause nephrosis. Phenothiazines are a class of psychoactive drugs that are used to treat schizophrenia, paranoia, mania, hyperactivity in children, some forms of senility, and anxiety (http://www.encyclopedia.com/articlesnew/36591.html). Side effects associated with prolonged use of these drugs are reduced blood pressure, Parkinsonism, reduction of motor activity, and visual impairment.

The present inventors have noted indications of liver and renal effects of AY-25329 by changes in serum chemistry. As early as 6 hours after the first dose, statistically significant increases in serum levels of creatinine, BUN, ALT, triglycerides and cholesterol were observed. Most of these markers of renal and liver dysfunction remained altered throughout the 14 day study period. Light microscopic analysis revealed effects in the liver as early as 6 and 24 hours, as evidenced by an increased number of hepatocytic mitotic figures and decreased glycogen content. Following 14 days of repeated dosing, nephrosis and alterations in the peripheral lobes of the liver and in the cytoplasm of hepatocytes were evident in rats dosed with 250 mg/kg/day of AY-25329.

BI liver toxin, a model compound, produces cardiac changes (QT_{C} prolongation) in dogs and liver and cardiac changes in rats. Liver samples collected from rats over a four-week period showed that this compound induces sedation, lowers body weight, increases liver weight, and slightly increases serum levels of AST, ALP and BUN. Over a three-month period, cardiovascular effects are observed as well.

The toxicological profile of bicalutamide, a drug for treating prostate-cancer, is closely associated with the drug's non-steroidal anti-androgenic activity. Bicalutamide produces typical effects of an anti-androgen, including atrophy of the prostate, testis and seminal vesicles and Leydig cell hyperplasia resulting from inhibition of pituitary feedback by testosterone. Benign Leydig cell tumors and elevated levels of CYP3A1 were seen in rats, but not in humans, although liver toxicity in humans has been observed. Bicalutamide causes liver enlargement and is a mixed function oxidase inducer in rodents and dogs. These effects lead to thyroid hypertrophy and adenoma in the rat and hepatocellular carcinoma in the male mouse (Iswaran et al. (1997) J Toxicol Sci 22(2):75-88; Oh et al. (2002) Urology 60(3 Suppl 1):87-93; McKillop et al. (1998) Xenobiotica 28(5):465-478). In prostate cancer patients treated with bicalutamide, elevated levels of the liver enzymes glutamic-oxalacetic transaminase (GOT), glutamic-pyruvic transaminase (GPT), alkaliphosphatase (AL-P) and gamma guanosine 5′-triphosphate (gamma-GTP) have been noted, along with breast pain, gynecomastia and hot flashes (Kotake et al. (1996) Hinyokika Kiyo 42(2):143-153).

The pathogenesis of acute carbon tetrachloride (CCl₄)-induced hepatotoxicity follows a well-characterized course in humans and experimental animals resulting in centrilobular necrosis and steatosis, followed by hepatic regeneration and tissue repair. Severity of the hepatocellular injury is also dose-dependent and may be affected by species, age, gender and diet.

Differences in susceptibility to CCl₄ hepatotoxicity are primarily related to the ability of the animal model to metabolize CCl₄ to reactive intermediates. CCl₄-induced hepatotoxicity is dependent on CCl₄ bioactivation to trichloromethyl free radicals by cytochrome P450 enzymes (CYP2E1), localized primarily in centrizonal hepatocytes. Formation of the free radicals leads to membrane lipid peroxidation and protein denaturation resulting in hepatocellular damage or death.

The onset of hepatic injury is rapid following acute administration of CCl₄ to male rats. Morphologic studies have shown cytoplasmic accumulation of lipids in hepatocytes within 1 to 3 hours of dosing, and by 5 to 6 hours, focal necrosis and hydropic swelling of hepatocytes are evident. Centrilobular necrosis and inflammatory infiltration peak by 24 to 48 hours post dose. The onset of recovery is also evident within this time frame by increased DNA synthesis and the appearance of mitotic figures. Removal of necrotic debris begins by 48 hours and is usually completed by one week, with full restoration of the liver by 14 days.

Increases in serum transaminase levels also parallel CCl₄-induced hepatic histopathology. In male Sprague Dawley (SD) rats, alanine aminotrasferase (ALT) and aspartate aminotransferase (AST) levels increase within 3 hours of CCl₄ administration (0.1, 1, 2, 3, 4 mL/kg, ip; 2.5 mL/kg, po) and reach peak levels (approximately 5-10 fold increases) within 48 hours post dose. Significant increases in serum α-glutathione s-transferase (α-GST) levels have also been detected as early as 2 hours after CCl₄ administration (25 μL/kg, po) to male SD rats.

At the molecular level, induction of the growth-related proto-oncogenes, c-fos and c-jun, is reportedly the earliest event detected in an acute model of CCl₄-induced hepatotoxicity (Schiaffonato et al. (1997) Liver 17:183-191). Expression of these early-immediate response genes has been detected within 30 minutes of a single dose of CCl₄ to mice (0.05-1.5 mL/kg, ip) and by 1 to 2 hours post dose in rats (2 mL/kg, po; 5 mL/kg,po) (Schiaffonato et al., supra, and Hong et al. (1997) Yonsei Medical J 38:167-177). Similarly, hepatic c-myc gene expression is increased by 1 hour following an acute dose of CCl₄ to male SD rats (5 mL/kg, po) (Hong et al., supra). Expression of these genes following exposure to CCl₄ is rapid and transient. Peak hepatic mRNA levels for c-fos, c-jun, and c-myc, after acute administration of CCl₄ have been reported at 1 to 2 hours, 3 hours, and 1 hour post dose, respectively.

The expression of tumor necrosis factor-α (TNF-α) is also increased in the livers of rodents exposed to CCl₄, and TNF-α has been implicated in initiation of the hepatic repair process. Pre-treatment with anti-TNF-α antibodies has been shown to prevent CCl₄-mediated increases in c-jun and c-fos gene expression, whereas administration of TNF-α induced rapid expression of these genes (Bruccoleri et al. (1997) Hepatol 25:133-141). Up-regulation of transforming growth factor-β (TGF-β) and transforming growth factor receptors (TBRI-III) later in the repair process (24 and 48 hours after CCl₄ administration) suggests that TGF-β may play a role in limiting the regenerative response by induction of apoptosis (Grasl-Kraupp et al. (1998) Hepatol 28:717-7126).

Chloroform (CHCl₃) is an obsolete anesthetic that was abandoned due to its hepatotoxicity. The pathogenesis of acute CHCl₃-induced hepatotoxicity follows a well-characterized course in humans and experimental animals resulting in centrilobular necrosis and steatosis, followed by hepatic regeneration and tissue repair. Severity of the hepatocellular injury is dose-dependent and may be affected by the animal species, strain, age, gender, diet, vehicle and/or route of administration (Lilly et al. (1997) Fund Appl Toxicol 40:101-110 and Raymond et al. (1997) J Toxicol Environ Health 52:463-476).

Differences in susceptibility to CHCl₃ toxicity are considered related to differential metabolism. CHCl₃-induced hepatotoxicity is primarily mediated by formation of reactive species, such as phosgene and trichloromethyl free radicals, by cytochrome P450 enzymes (CYP2E1). CHCl₃ hepatotoxicity is also increased by exposure to agents that induce cytochrome P450 (i.e., ethanol, phenobarbital), and deplete hepatic glutathione (GSH). Formation of the free radicals leads to membrane lipid peroxidation and protein denaturation resulting in hepatocellular damage or death.

Chronic administration of CHCl₃ to rodents induces an increased incidence of hepatic and renal carcinomas by a nongenotoxic-cytotoxic mode of action. Carcinogenicity of CHCl₃ is considered secondary to chemically-induced cytotoxicity with subsequent compensatory cell proliferation, rather than to direct interaction of CHCl₃ or its metabolites with DNA.

The onset of hepatic toxicity is rapid following acute administration of CHCl₃ to male rats. Morphologic studies have shown cytoplasmic accumulation of lipids in hepatocytes within 1 to 3 hours of dosing, and by 5 to 6 hours, focal necrosis and hydropic swelling of hepatocytes are evident. Centrilobular necrosis and inflammatory infiltration peak by 24 to 48 hours post dose. The onset of recovery is also evident within this time frame by increased DNA synthesis and the appearance of mitotic figures. Removal of necrotic debris begins by 48 hours and is usually completed by one week, with full restoration of the liver by 14 days.

In studies on rats and mice, significant changes in clinical parameters included increased levels of BUN and serum creatinine and decreased levels of phosphatidyl-ethanolamine and tissue glutathione (GSH). There is a strong correlation between the formation of the phospholipid adducts, GSH depletion and liver toxicity (Di Consiglio et al. (2001) Toxicology 159(1-2):43-53). Experiments on mice have shown that exposure to chloroform also increases the liver weight:body weight ratio and the proliferating cell nuclear antigen-labeling index. Decreased levels of 5-methylcytosine and of the methylated c-myc gene (associated with increased carcinogenic activity) were also found (Coffin et al. (2000) Toxicol Sci 58(2):243-252). Other studies on mice have noted that elevated levels of the P450 cytochromes, such as P450 2E1 and CYP2A5, are involved in cytotoxic metabolic conversions (Constan et al. (1999) Toxicol Appl Pharmacol 160(2):120-126; Camus-Randon et al. (1996) Toxicol Appl Pharmacol 138(1):140-148).

Studies of chloroform poisoning in humans have noted hepatocellular necrosis characterized by decreased levels of serum biomarkers (AST, ALT, alkaline phosphatase and lactate dehydrogenase) and increased levels of markers of hepatocellular regeneration (alpha-fetoprotein, retinol-binding protein, gamma-glutamyl transferase and des-gamma-carboxyprothrombin) (Horn et al. (1999) Am J Clin Pathol 112(3):351-357).

At the molecular level, CHCl₃-induced changes in mRNA levels of 2 known genes, MUSTI21 (a mouse primary response gene induced by growth factors and tumor promoters) and MUSMRNAH (a gene highly homologous to a gene isolated from a prostate carcinoma cell line), and 2 novel genes (MUSFRA and MUSFRB) have been identified by differential display in regenerating mouse liver (Kegelmeyer et al. (1997) Molecul Carcin 20:288-297). These genes have been postulated to play a role in hepatic regeneration or possibly CHCl₃-induced hepatocarcinogenesis.

CI-1000 (4H-pyrrolo:3,2-d:pyrimidin-4-one, 2-amino-3,5-dihydro-7-(3-thienylmethyl)-monohydrochloride monohydrate) is a compound with anti-inflammatory properties. After treatment with CI-1000, increased serum ALT levels, a standard marker of liver toxicity, were observed in dogs.

Clofibrate, a halogenated phenoxypropanoic acid derivative (ethyl ester of clofibric acid), is an antilipemic agent. The exact mechanism by which clofibrate lowers serum concentrations of triglycerides and low-density lipoprotein (LDL) cholesterol, as well as raising high-density lipoprotein (HDL) cholesterol is uncertain. The drug has several antilipidemic actions, including activating lipoprotein lipase, which enhances the clearance of triglycerides and very-low-density lipoprotein (VLDL) cholesterol, inhibition of cholesterol and triglyceride biosynthesis, mobilization of cholesterol from tissues, increasing fecal excretion of neutral steroids, decreasing hepatic lipoprotein synthesis and secretion, and decreasing free fatty acid release.

Clofibrate has a number of effects on the rat liver, including hepatocellular hypertrophy, cellular proliferation, hepatomegaly, induction of CYP450 isozymes, and induction of palmitoyl CoA oxidation. Long term administration of clofibrate causes increased incidence of hepatocellular carcinoma, benign testicular Leydig cell tumors, and pancreatic acinar adenomas in rats. Clofibrate induces proliferation of peroxisomes in rodents and this effect, rather than genotoxic damage, is believed to be the causative event in rodent carcinogenesis (AHFS Drug Information Handbook 2001, McEvoy, ed., pp. 1735-1738; Electronic Physicians' Desk Reference-Atromid-S (clofibrate) at www.pdr.net; Brown and Goldstein, “Drugs used in the treatment of hyperliproteinemias,” in Goodman and Gilman's The Pharmacological Basis of Therapeutics, Eighth ed., Goodman et al., eds., pp. 874-896, Pergamon Press, New York, 1990).

Clofibrate also increases hepatic lipid content and alters its normal composition by significantly increasing levels of phosphatidylcholine and phosphatidyl-ethanolamine (Adinehzadeh et al. (1998) Chem Res Toxicol 11(5):428-440). A rat study of liver hyperplasia and liver tumors induced by peroxisome proliferators revealed that administration of clofibrate increased levels of copper and altered copper-related gene expression in the neoplastic liver tissues. Down-regulation of the ceruloplasmin gene and of the Wilson's Disease gene (which encodes P-type ATPase), along with up-regulation of the metallothionein gene, were noted in these tissues (Eagon et al. (1999) Carcinogenesis 20(6): 1091-1096). Clofibrate-induced peroxisome proliferation and carcinogenicity are believed to be rodent-specific, and have not been demonstrated in humans.

Colchicine, an alkoloid of Colchicum autumale, is an antiinflammatory agent used in the treatment of gouty arthritis (Goodman & Gilman's The Pharmacological Basis of Therapeutics 9^{th} ed., p. 647, J. G. Hardman et al., Eds., McGraw Hill, New York, 1996). An antimitotic agent, colchicine binds to tubulin which leads to depolymerization and disappearance of the fibrillar microtubules in granulocytes and other motile cells. As a result, the migration of granulocytes into the inflamed area is inhibited, thereby suppressing the inflammatory response.

Some common, mild side effects associated with colchicine treatment are gastrointestinal disturbances, loss of appetite and hair loss. More serious side effects include hepatotoxicity, nausea, vomiting, and severe diarrhea and/or abdominal pain. Colchicine overdose can induce convulsions, coma, and multiorgan failure with a high incidence of mortality. Renal failure is multifactorial and related to prolonged hypotension, hypoxemia, sepsis, and rhabdomyolysis. In rats, less dramatic doses have been shown to inhibit the secretion of many endogenous proteins such as insulin and parathyroid hormone. Signs of liver damage are leakage of marker compounds, such as lactate dehydrogenase and albumin, into plasma and bile (Dvorak et al. (2002) Toxicol In Vitro 16(3):219-227; Crocenzi et al. (1997) Toxicology 121(2):127-142).

Cyproterone acetate (CPA) is a potent androgen antagonist and has been used to treat acne, male pattern baldness, precocious puberty, and prostatic hyperplasia and carcinoma (Goodman & Gilman's The Pharmacological Basis of Therapeutics 9^{th} ed., p. 1453, J. G. Hardman et al., Eds., McGraw Hill, New York, 1996). Additionally, CPA has been used clinically in hormone replacement therapy to protect the endometrium and decrease menopausal symptoms and the risk of osteoporotic fracture (Schneider, “The role of antiandrogens in hormone replacement therapy,” Climacteric 3 (Suppl. 2): 21-27 (2000)).

In experiments with rats, CPA was shown to induce unscheduled DNA synthesis in vitro. After a single oral dose, continuous DNA repair activity was observed after 16 hours. CPA also increased the occurrence of S phase cells, which corroborated the mitogenic potential of CPA in rat liver (Kasper et al. (1996) Carcinogenesis 17(10): 2271-2274). CPA has also been shown to produce cirrhosis in humans (Garty et al. (1999) Eur J Pediatr 158(5): 367-370).

Diclofenac, a non-steroidal anti-inflammatory drug, has been frequently administered to patients suffering from rheumatoid arthritis, osteoarthritis, and ankylosing spondylitis. Following oral administration, diclofenac is rapidly absorbed and then metabolized in the liver by cytochrome P450 isozyme of the CYC2C subfamily (Goodman & Gilman's The Pharmacological Basis of Therapeutics 9^{th} ed., p. 637, J. G. Hardman et al., eds., McGraw Hill, New York, 1996). In addition, diclofenac has been applied topically to treat pain due to corneal damage (Jayamanne et al., (1997) Eye 11(Pt. 1): 79-83; Domic et al. (1998) Am J Ophthalmol 125(5): 719-721).

Although diclofenac has numerous clinical applications, adverse side-effects have been associated with the drug, such as corneal complications, including corneal melts, ulceration, and severe keratopathy (Guidera et al. (2001) Ophthalmology 108(5): 936-944). Another study investigated 180 cases of patients who had reported adverse reactions to diclofenac to the Food and Drug Administration (Banks et al. (1995) Hepatology 22(3): 820-827). Of the 180 reported cases, the most common symptom was jaundice (75% of the symptomatic patients). Liver sections were taken and analyzed, and hepatic injury was apparent one month after drug treatment. An additional report showed that a patient developed severe hepatitis five weeks after beginning diclofenac treatment for osteoarthritis (Bhogaraju et al. (1999) South Med J 92(7): 711-713).

In one study on diclofenac-treated Wistar rats (Ebong et al. (1998) Afr J Med Sci 27(3-4): 243-246), diclofenac treatment induced an increase in serum chemistry levels of alanine aminotransferase, aspartate aminotransferase, methaemoglobin, and total and conjugated bilirubin. Additionally, diclofenac enhanced the activity of alkaline phosphatase and 5′nucleotidase. A study on humans revealed elevated levels of hepatic transaminases and serum creatine when compared to the control group (McKenna et al. (2001) Scand J Rheumatol 30(1): 11-18).

Diflunisal, a non-steroidal anti-inflammatory drug (NSAID), is a difluorophenyl derivative of salicylic acid (Goodman & Gilman's The Pharmacological Basis of Therapeutics 9^{th} ed., p. 631, J. G. Hardman et al., Eds., McGraw Hill, New York, 1996). It is most frequently used in the treatment of osteoarthritis and musculoskeletal strains. NSAIDs have analgesic, antipyretic and anti-inflammatory actions, however, hepatotoxicity is known to be an adverse side effect of NSAID treatment (Masubuchi et al. (1998) J Pharmacol Exp Ther 287:208-213). Diflunisal has been shown to be less toxic than other NSAIDs, but it can eventually have deleterious effects on platelet or kidney function (Bergamo et al. (1989) Am J Nephrol 9:460-463). Other side effects that have been associated with diflunisal treatment are diarrhea, dizziness, drowsiness, gas or heartburn, headache, nausea, vomiting, and insomnia (http://arthritisinsight.com/medical/meds/dolobid.html).

In a comparative hepatotoxicity study of 18 acidic NSAIDs, diflunisal was shown to increase LDH leakage in rat hepatocytes, a marker for cell injury, when compared to control samples. Additionally, treatment with diflunisal led to decreased intracellular ATP concentrations. In a study comparing the effects of diflunisal and ibuprofen, (Muncie and Nasrallah (1989) Clin Ther 11:539-544) both drugs appeared to cause abdominal cramping, even during short-term usage. Because the toxic dosages were selected to be below the level at which gastric ulceration occurs, more severe gastrointestinal effects were not detected. But, increased serum levels of creatinine, a sign of renal injury, were also observed (Muncie et al. (1989) Clin Ther 11:539-544).

Dioxin, an environmental and workplace toxin, is the name given to a class of compounds that are bi-products in the manufacture of chlorinated herbicides, pesticides and plastics. The most toxic and carcinogenic of these is 2,3,7,8-tetrachlorodibenzo-p-dioxin (2,3,7,8-TCDD). Exposure to dioxin increases expression of the aromatic hydrocarbon (Ah) receptor and also increases the production of reactive oxygen species in the mitochondria. Dioxin also increases mitochondrial levels of CYP1A1, CYP1A2 and glutathione, as well as hepatocyte levels of SOD and enzymes associated with oxidative stress (Senft et al. (2002) Free Radic Biol Med 33: 1268-1278; Kern et al. (2002) Toxicology 171: 117-1125.

Another model compound, dimethylnitrosamine (DMN), is a known carcinogen and inducer of liver fibrosis and lipid peroxidation. DMN causes oxidative stress in liver cells, which may be the link between chronic liver damage and liver fibrosis. Rats treated with DMN showed diffuse fibronectin deposition, elevated hydroxyproline levels (an indicator of fibrosis), increased levels of collagens, fibrous septa, and impaired oxidative balance. Serum levels of ALT and malondialdehyde (MDA) were increased, while serum levels of SOD were decreased (Vendemiale et al. (2001) Toxicol Appl Pharmacol 175: 130-139; Liu et al. (2001) Zhonghua Gan Zang Bing Za Zhi 9 Suppl: 18-20). Other studies in rats have noted severe centrilobular congestion and haemorrhagic necrosis several days after a three-day period of DMN administration. Following additional periods of DMN treatment, the rats developed centrilobular necrosis and intense neutrophilic infiltration, which progressed to severe centrilobular necrosis, fiber deposition, focal fatty deposits, bile duct proliferation, bridging necrosis and fibrosis around the central veins (cirrhosis-like symptoms). A decrease in total protein and increase in DNA were also observed (George et al. (2001) Toxicology 156: 129-138).

17α-ethinylestradiol, a synthetic estrogen, is a component of oral contraceptives, often combined with the progestational compound norethindrone. It is also used in post-menopausal estrogen replacement therapy (PDR 47^{th} Ed., pp. 2415-2420, Medical Economics Co., Inc., Montvale, N.J., 1993; Goodman & Gilman's The Pharmalogical Basis of Therapeutics 9^{th} Ed., pp. 1419-1422, J. G. Hardman et al. Eds., McGraw Hill, New York, 1996).

The most frequent adverse effects of 17α-ethinylestradiol usage are increased risks of cardiovascular disease: myocardial infarction, thromboembolism, vascular disease and high blood pressure, and of changes in carbohydrate metabolism, in particular, glucose intolerance and impaired insulin secretion. There is also an increased risk of developing benign hepatic neoplasia. Because this drug decreases the rate of liver metabolism, it is cleared slowly from the liver, and carcinogenic effects, such as tumor growth, may result.

17α-ethinylestradiol has been shown to cause a reversible intrahepatic cholestasis in male rats, mainly by reducing the bile-salt-independent fraction of bile flow (BSIF) (Koopen et al. (1998) Hepatology 27: 537-545). Plasma levels of bilirubin, bile salts, aspartate aminotransferase (AST) and alanine aminotransferase (ALT) in this study were not changed. This study also showed that 17α-ethinylestradiol produced a decrease in plasma cholesterol and plasma triglyceride levels, but an increase in the weight of the liver after 3 days of drug administration, along with a decrease in bile flow. Further results from this study are as follows. The activities of the liver enzymes leucine aminopeptidase and alkaline phosphatase initially showed significant increases, but enzyme levels decreased after 3 days. Bilirubin output increased, although glutathione (GSH) output decreased. The increased secretion of bilirubin into the bile without affecting the plasma level suggests that the increased bilirubin production must be related to an increased degradation of heme from heme-containing proteins. Similar results were obtained in another experiment (Bouchard et al., (1993) Liver 13: 193-202) in which the livers were also examined by light and electron microscopy. Daily doses of 17α-ethinylestradiol have been shown to cause cholestasis as well, although, following drug treatment, bile flow rates gradually returned to normal (Hamada et al. (1995) Hepatology 21: 1455-1464). Liver hyperplasia, possibly in response to the effects of tumor promoters, has also been observed (Mayol (1992) Carcinogenesis 13: 2381-2388).

The lipid-lowering drug gemfibrozil is a know peroxisome proliferator in liver tissue, causing both hyperplasia and enlargement of liver cells. Upon exposure to gemfibrozil, hepatocarcinogenesis has been observed in rats and mice, and a decrease in alpha-tocopherol and an increase in DT-diaphorase activity have been observed in rats and hamsters (impaired antioxidant capability). Peroxisome proliferators increase the activities of enzymes involved in peroxisomal beta-oxidation and omega-hydroxylation of fatty acids, which results in oxidative stress (O'Brien et al. (2001) Toxicol Sci 60: 271-278; Carthew et al. (1997) J Appl Toxicol 17: 47-51).

Hydrazine (NH₂═NH₂), is a component of many industrial chemicals, such as aerospace and airplane fuels, corrosion inhibitors, dyes and photographic chemicals. Its derivatives are used in pharmaceuticals such as hydrazine sulphate, used to treat cachexia in cancer patients, isoniazid, an anti-tuberculosis drug, and hydralazine, an anti-hypertensive. These drugs are metabolized in vivo to produce hydrazine, among other by-products. Consequently, exposure to hydrazine is by direct contact, e.g., among military and airline personnel, or the result of its production in the body, e.g., in patients with cancer or high blood pressure.

Studies on rat hepatocytes have shown that hydrazine causes a dose-dependent loss of viability, leakage of LDH, depletion of GSH and ATP and a decreased rate of protein synthesis (Delaney et al. (1995) Xenobiotica 25: 1399-1410). When administered to rats, hepatotoxic changes, characterized by GSH and ATP depletion and induction of fatty liver (increases in liver weight and triglycerides, with the appearance of fatty droplets, swelling of mitochondria and appearance of microbodies) were also found to be dose-dependent (Jenner et al. (1994) Arch Toxicol 68: 349-357; Scales et al. (1982) J Toxicol Environ Health 10: 941-953). The hepatoxicity, as well as renal toxicity, associated with hydrazine exposure has been linked to free radical damage resulting from oxidative metabolism by cytochrome P4502E1 (CYP2E1), which catalyzes the conjugation of free radicals with reduced glutathione. Although exposure to hydrazine and several hydrazine derivatives increased enzyme levels in kidney tissue, increased enzyme levels were not detected in liver tissue (Runge-Morris et al. (1996) Drug Metab Dispos 24: 734-737).

The mutagenic and hepatocarcinogenic effects of hydrazine were examined in hamster livers. In vivo, hydrazine reacts with formaldehyde to form formaldehyde hydrazone (CH₂═N—NH₂), an alkylating intermediate that methylates guanine in DNA. Upon treatment with hydrazine, liver DNA showed the presence of methylated guanine, DNA adducts and the impairment of maintenance methylation (impaired methylation of deoxycytosine). Hepatic adenomas and carcinomas also developed in a dose-dependent manner and could be correlated with decreased maintenance methylation (FitzGerald et al. (1996) Carcinogenesis 17: 2703-2709).

Indomethacin is a non-steroidal antiinflammatory, antipyretic and analgesic drug commonly used to treat rheumatoid arthritis, osteoarthritis, ankylosing spondylitis, gout and a type of severe, chronic cluster headache characterized by many daily occurrences and jabbing pain. This drug acts as a potent inhibitor of prostaglandin synthesis; it inhibits the cyclooxygenase enzyme necessary for the conversion of arachidonic acid to prostaglandins (PDR 47^{th} Ed., Medical Economics Co., Inc., Montvale, N.J., 1993; Goodman & Gilman's The Pharmalogical Basis of Therapeutics 9^{th} Ed., J. G. Hardman et al. eds., pp. 1074-1075, 1089-1095, McGraw Hill, New York, 1996; Cecil Textbook of Medicine, 20^{th} Ed., part XII, pp. 772-773, 805-808, J. C. Bennett and F. Plum Eds., W.B. Saunders Co., Philadelphia, 1996).

The most frequent adverse effects of indomethacin treatment are gastrointestinal disturbances, usually mild dyspepsia, although more severe conditions, such as bleeding, ulcers and perforations can occur. Hepatic involvement is uncommon, although some fatal cases of hepatitis and jaundice have been reported. Renal toxicity can also result, particularly after long-term administration. Renal papillary necrosis has been observed in rats, and interstitial nephritis with hematuria, proteinuria and nephrotic syndrome have been reported in humans. Patients suffering from renal dysfunction risk developing a reduction in renal blood flow, because renal prostaglandins play an important role in renal perfusion.

In rats, although indomethacin produces more adverse effects in the gastrointestinal tract than in the liver, it has been shown to induce changes in hepatocytic cytochrome P450. In one study, no widespread changes in the liver were observed, but a mild, focal, centrilobular response was noted. Serum levels of albumin and total protein were significantly reduced, while the serum level of urea was increased. No changes in creatinine or aspartate aminotransferase (AST) levels were observed (Falzon et al. (1985) Br J exp Path 66: 527-534). In another rat study, a single dose of indomethacin was shown to reduce liver and renal microsomal enzymes, including CYP450, and cause lesions in the GI tract (Fracasso et al. (1990) Agents Actions 31: 313-316).

Menadione (vitamin K₃) is a fat-soluble vitamin precursor that is converted into menaquinone in the liver. The primary known function of vitamin K is to assist in normal blood clotting, but it may also play a role in bone calcificaton. Menadione is a quinone compound that induces oxidative stress. It has been used as an anticancer agent and radiosensitizer and can produce toxicity in the kidney, lung, heart, and liver. In the kidney, signs of toxicity are dose-dependent, ranging from minor degranulation of tubular cells at lower doses to tubular dilatation, formation of protein casts in the renal tubules, calcium mineralization, vacuolization in the proximal and distal renal tubules, granular degeneration in the cortex and necrosis and apoptosis (Chiou et al. (1997) Toxicology 124: 193-202). Toxic effects in the liver include depletion of glutathione, increased levels of Ca²⁺, increased lipid peroxidation and protein thiol oxidation, DNA strand breaks, and plasma membrane protrusions (blebs), which lead to cell degeneration. Oxidative stress induced by menadione also causes cytoskeletal abnormalities, which are related to the surface blebs (Chiou et al. (1998) Proc Natl Sci Counc Repub China B 22: 13-21; Mirabelli et al. (1988) Arch Biochem Biophys 264: 261-269).

Phenobarbital is used as an anti-epileptic, sedative or hypnotic drug and can also be used to treat neuroses with related tension states, such as hypertension, coronary artery disease, gastrointestinal disturbances and preoperative apprehension. Phenobarbital is also found in medications to treat insomnia and headaches (Remington: The Science and Practice of Pharmacy, 19th Ed., A. R. Gennaro ed., pp. 1164-1165, Mack Publishing Co., Easton, Pa., 1995). Although liver toxicity is not a common side effect, the drug produces elevated levels of CYP2B1, and incidences of cholestasis and hepatocellular injury have been found (Selim et al. (1999) Hepatology 29: 1347-135; Gut et al. (1996) Environ Health Perspect 104: 1211-1218).

Tacrine (1,2,3,4-tetrahydro-9-aminoacridine-hydrochloride), a strong acetylcholinesterase (AChE) inhibitor, is used in the treatment of mild to moderate cases Alzheimer's dimentias. Alzheimer's patients have synaptic loss, neuronal atrophy and degeneration of cholinergic nuclei in the forebrain, which are associated with reduced oxidative metabolism of glucose and decreased levels of ATP and acetyl CoA. Administration of AChE inhibitors, such as tacrine, is designed to increase cholinergic activity to combat this loss (Weinstock (1995) Neurodegeneration 4: 349-356). The effect seen in the patients is a reversal of the cognitive and functional decline, but the drug does not appear to change the neurodegenerative process (Goodman & Gilman's The Pharmacological Basis of Therapeutics 9^{th} Ed., Hardman et al. eds., p. 174, McGraw Hill, New York, 1996).

Hepatotoxicty caused by tacrine is typically reversible, although cases of severe hepatotoxicity have been seen (Blackard et al. (1998) J Clin Gastroenterol 26: 57-59). The toxicity is characterized by decreased levels of protein synthesis and the release of lactate dehydrogenase, as well as by increased transaminase levels and decreased levels of ATP, glycogen and glutathione. The decrease in protein synthesis may represent a signal leading to cell death (Lagadic-Gossmann et al. (1998) Cell Biol Toxicol 14: 361-373).

Preclinical studies have failed to detect adverse hepatic events, although tacrine displayed cytotoxicity to human hepatoma cell lines and primary rat hepatocytes (Viau et al. (1993) Drug Chem Toxicol 16: 227-239). While hepatotoxicity has been found in humans, in vivo rat studies have not shown a correlation between tacrine exposure and hepatotoxicity, and the mechanism of action is not completely understood. An in vitro study comparing the reaction of human and rat liver microsomal preparations to tacrine (Woolf et al. (1993) Drug Metab Dispos 21: 874-882) showed that the two species reacted differently to the drug, suggesting that the rat may not be the best model for monitoring tacrine-induced elevations in liver marker enzymes (Woolf et al. (1993) Drug Metab Dispos 21: 874-882).

While tacrine does not reveal classic signs of hepatotoxicity in rats, gene expression changes due to tacrine administration can be used to predict that the drug will be a liver toxin in humans. This suggests that toxicogenomics might be able to detect drugs that prove to be toxic in the clinic even when classical but more crude measures in preclinical screening fail to detect toxicity.

Thioacetamide's only significant commercial use is as a replacement for hydrogen sulfide in qualitative analyses (IARC, Vol. 7, 1974). It has also been used as a fungicide, an organic solvent in the leather, textile and paper industries, as an accelerator in the vulcanization of buna rubber, and as a stabilizer of motor fuel. The primary routes of human exposure are inhalation and skin contact with products in which thioacetamide was used as a solvent (9th Report on Carcinogens, U.S. Dept. of Health and Human Services, Public Health Service, National Toxicology Program, http://ehp.niehs.nih.gov/roc/toc9.html). Thioacetamide is metabolized to a nonionic electrophile, leading to oxidative stress and other injurious events; both cytochrome P4502E1 and the flavin-containing monooxygenase system have been implicated in this bioactivation (R. Snyder & L. S. Andrews, Toxic Effects of Solvents and Vapors, in Casarett & Doull's Toxicology: The Basic Science of Poisons, Klaasen, ed., p. 737, McGraw-Hill, New York, 1996; Smith et al. (1983) Toxicol Appl Pharmacol 70: 467-479; Jurima-Romet et al. (1993) Biochem Pharmacol 14:46(12):2163-2170).

In exposed rats, thioacetamide was shown to accumulate in the liver and kidney, resulting in elevated levels of serum total bilirubin, aspartate aminotransferase, alanine aminotransferase, BUN, creatinine and TNFα. Impaired clearance of the toxin and increased secretion of TNFα are related to the progression of toxic effects in the liver and kidney (Nakatani et al. (2001) Liver 21(1):64-70). Additional histological changes in kidney tissue include glomerular tuft collapse and interstitial haemorrhage (Caballero et al. (2001) Gut 48: 34-40).

In the liver, low acute doses of thioacetamide induce apoptosis, while high acute doses induce necrosis (Casarett & Doull's Toxicology, supra). Long term exposure induces cirrhosis and tumors (Risteli et al. (1976) Biochem J 158: 361-367). The acute liver injury is characterized by severe penvenous necrosis, immediately followed by hepatocellular regeneration and this necrosis. Nitric oxide synthase activity and nitric oxide release are thought to play a role in the pathophysiological mechanisms that trigger liver regeneration following thioacetamide exposure (Ala-Kokko et al. (1987) Biochem J 244: 75-79). Exposure to thioacetamide also decreases levels of antioxidants, such as SOD, glutathione peroxidase and uric acid. It also increases apoptosis, along with caspase-3 activity, and has been observed to affect hepatic nitrogen metabolism. Rates of urea production and excretion were decreased, as well as glutamate dehydrogenase activity and glutamine synthetase activity. Mitogenic activity and DNA synthesis, however, were observed to increase (Abul et al. (2002) Anat Histo Embryol 31: 66-71; Hayami et al. (1999) Biochem Pharmacol 58: 1941-1943; Masumi et al. (1999) Toxicology 135: 21-31; Maier et al. (1991) Arch Toxicol 65: 454-464).

Valproate (n-dipropylacetic acid, Depakene®) is routinely used to treat several types of epileptic seizures-absence seizures, myoclonic seizures and tonic-clonic seizures. Most other anti-epileptics are effective against only one type. Valproate acts on neurons to inhibit the sustained repetitive firing caused by depolarization of cortical or spinal cord neurons, and a prolonged recovery of inactivated voltage-activated Na⁺ channels follows. The drug also acts by reducing the low-threshold Ca²⁺ current and its multiple mechanisms contribute to its use in multiple types of seizures. Although valproate does not affect neuronal responses to GABA, it does increase the activity of the GABA synthetic enzyme, glutamic acid decarboxylase, and it inhibits enzymes that degrade GABA, GABA transaminase and succinic semialdehyde dehydrogenase (Goodman and Gilman's The Pharmacological Basis of Therapeutics. 9th Ed., Hardman et al., eds., pp. 462, 476 and 477, McGraw-Hill, New York, 1996).

The most common side effects are gastrointestinal symptoms, including anorexia, nausea and vomiting. Effects on the CNS include sedation, ataxia and tremor. Rash, hair loss, increased appetite and teratogenic effects have also been observed (Briggs et al., A Reference Guide to Fetal and Neonatal Risk. Drugs in Pregnancy and Lactation. 4th ed., p. 869, Williams & Wilkins, Baltimore, 1994). With respect to liver toxicity, valproate produces elevated levels of hepatic enzymes in about 40% of patients, which may be an asymptomatic condition, and elevated levels of hepatic lipids. Fulminant hepatitis, microvesicular steatosis (fatty degeneration), hepatocyte necrosis and hepatic failure can also result. It is believed that hepatoxicity is caused by an accumulation of unsaturated metabolites of valproate, in particular 4-en-valproate, which is structurally similar to two known hepatotoxins, 4-en-pentanoate and methylenecyclopropylacetic acid (Eadie et al. (1988) Med Toxicol Adverse Drug Exp 3: 85-106).

In a study on rats, microvesicular steatosis caused by valproate was found to be accompanied by myeloid bodies, lipid vacuoles and mitochondrial abnormalities (Kesterson et al. (1984) Hepatology 4: 1143-1152). Experiments on cultured rat hepatocytes have shown that valproate produces a dose-dependent leakage of lactic acid dehydrogenase and increased amounts of acyl-CoA esters, compounds that interfere with the beta-oxidation of fatty acids (Vance et al. (1994) Epilepsia 35: 1016-1022). Administration of valproate to rats has also been shown to cause enhanced excretion of dicarboxylic acids, a sign of impaired mitochondrial beta-oxidation. Other metabolic effects include hypoglycemia, hyperammonemia, decreased levels of beta-hydroxybutyrate and carnitine and decreased activities of acyl-CoA dehydrogenases, enzymes involved in fatty acid oxidation. mRNA levels of genes involved in fatty acid oxidation, however, such as the short-, medium- and long-chain acyl-CoA dehydrogenases, were found to have increased (Kibayashi et al. (1999) Pediatr Int 41: 52-60).

Wy-14643, a tumor-inducing compound that acts in the liver, has been used to study the genetic profile of cells during the various stages of carcinogenic development, with a view toward developing strategies for detecting, diagnosing and treating cancers (Rockett et al. (2000) Toxicology 144(1-3):13-29). In contrast to other carcinogens, Wy-14643 does not mutate DNA directly. Instead, it acts on the peroxisome proliferator activated receptor-alpha (PPARalpha), as well as on other signaling pathways that regulate growth (Johnson et al. (2001) J Steroid Biochem Mol Biol 77(1):59-71). The effect is elevated and sustained cell replication, accompanied by a decrease in apoptosis (Rusyn et al. (2000) Carcinogenesis 21(12):2141-2145). These authors (Rusyn et al.) noted an increase in the expression of enzymes that repair DNA by base excision, but no increased expression of enzymes that do not repair oxidative damage to DNA. In a study on rodents, Johnson et al. noted that Wy-14643 inhibited liver-X-receptor-mediated transcription in a dose-dependent manner, as well as de novo sterol synthesis.

In experiments with mouse liver cells (Peters et al. (1998) Carcinogenesis 19(11):1989-1994), exposure to Wy-14643 produced increased levels of acyl CoA oxidase and proteins involved in cell proliferation: CDK-1, 2 and 4, PCNA and c-myc. Elevated levels may be caused by accelerated transcription that is mediated directly or indirectly by PPARalpha. It is likely that the carcinogenic properties of peroxisome proliferators are due to the PPARalpha-dependent changes in levels of cell cycle regulatory proteins.

Another study on rodents (Keller et al. (1992) Biochim Biophys Acta 1102(2):237-244) showed that Wy-14643 was capable of uncoupling oxidative phosphorylation in rat liver mitochondria. Rates of urea synthesis from ammonia and bile flow, two energy-dependent processes, were reduced, indicating that the energy supply for these processes was disrupted as a result of cellular exposure to the toxin. Wy-14643 has also been shown to activate nuclear factor kappaB, NADPH oxidase and superoxide production in Kupffer cells (Rusyn et al. (2000) Cancer Res 60(17):4798-4803). NADPH oxidase is known to induce mitogens, which cause proliferation of liver cells.

The anti-asthma drug zileuton is a 5-lipoxygenase inhibitior and leukotriene synthesis inhibitor and is given to asthma patients to counter the negative effects of leukotrienes-exacerbation of the harmful effects of the inflammatory process and bronchoconstriction. Zileuton has, however, been reported to cause hepatomegaly and elevated levels of liver peroxisomal palmitoyl CoA oxidase and microsomal cytochromes P450 2B and P450 4A. The monooxygenase activities of these cytochromes was also seen to increase (Rodrigues et al. (1996) Toxicol Appl Pharmacol 137(2):193-201; Sorkness (1997) Pharmacotherapy 17(1 Pt 2):50S-54S).

LPS (lipopolysaccharide) is an endotoxin released by gram-negative bacteria upon breakage or rupture of the cells that induces an acute inflammatory response in mammals and that can cause septic shock. LPS is also a research tool used to initiate liver injury in rats through an inflammatory mechanism. Typically, the membrane components of LPS are lipid-A, KDO (2-keto-3-deoxy-octulosonic acid), core polysaccharides and O-antigen polysaccharides, the polysaccharide units differing from one bacterium to another (Zinsser Microbiology 20th Ed., Joklik et al., eds., pp. 82-87, Appleton & Lange, Norwalk, Conn., 1992).

Primary rat hepatocytes derived from liver parenchymal cells and sinusoidal cells of rats that have been exposed to LPS in vivo can directly respond to LPS in cell culture. Numerous effects of LPS-induced endotoxemia can be detected, including elevated levels of nitric oxide synthetase (NOS) with increased nitric oxide and nitrite production, cellular hypertrophy, vacuolization, chromosomal emargination, cytoplasmic DNA fragmentation and necrosis (Pittner et al. (1992) Biochem Biophys Res Commun 185(1):430-435; Laskin et al., (1995) Hepatology 22(1):223-234; Wang et al. (1995) Am J Physiol 269(2 Pt 1):G297-304). Other studies have indicated that the presence of Kupffer cells with primary rat hepatocytes is essential for the induction of hepatocyte apoptosis by LPS (Hamada et al. (1999) J Hepatol 30(5):807-818).

Exposure of rats or primary hepatocytes to LPS induces the expression of a number of acute-phase proteins in the liver. Recent evidence has indicated that rat hepatocytes express soluble CD14 protein, and LPS is capable of markedly increasing levels of CD14 at both the gene expression and protein expression levels (Liu et al. (1998) Infect Immun 66(11):5089-5098). Soluble CD14 is believed to be a critical LPS recognition protein required for the activation of a variety of cells to toxic levels of LPS, even in endothelial and epithelial cells (Pugin et al. (1993) Proc Natl Acad Sci USA 90(7):2744-2748). Another key component of the LPS recognition system is lipopolysaccharide-binding protein (LBP), which binds to LPS. The LPS-LBP complex interacts with the CD14 receptor, inducing LPS sensitive genes. LBP can be induced in hepatocytes isolated from rats that have not been primed with LPS, indicating that this key regulatory pathway is intact in primary rat hepatocytes (Wan et al. (1995) Infect Immun 63(7):2435-2442).

Toxicity Prediction and Modeling

The genes and gene expression information, as well as the portfolios and subsets of the genes provided in Tables 1-5WWW, such as the core toxicity markers in Tables 5A-5WWW, may be used to predict at least one toxic effect, including the hepatotoxicity of a test or unknown compound. As used, herein, at least one toxic effect includes, but is not limited to, a detrimental change in the physiological status of a cell or organism. The response may be, but is not required to be, associated with a particular pathology, such as tissue necrosis. Accordingly, the toxic effect includes effects at the molecular and cellular level. Hepatotoxicity is an effect as used herein and includes but is not limited to the pathologies of liver necrosis, hepatitis, steatosis (fatty degeneration of the liver), carcinogenesis, cholestasis, liver enlargement, inflammation and peroxisome proliferation.

In general, assays to predict the toxicity or hepatotoxicity of a test agent (or compound or multi-component composition) comprise the steps of exposing a cell population to the test compound, assaying or measuring the level of relative or absolute gene expression of one or more of the genes in Tables 1-5WWW and comparing the identified expression level(s) to the expression levels disclosed in the Tables and database(s) disclosed herein. Assays may include the measurement of the expression levels of about 2, 3, 4, 5, 6, 7, 8, 9, 10, 15, 20, 25, 30, 50, 75, 100 or more genes from Tables 1-5WWW to create multi-gene expression profiles. In some instances, expression levels are assayed and compared for and to all or substantially all the genes in the tables.

In the methods of the invention, the gene expression level for a gene or genes induced by the test agent, compound or compositions may be comparable to the levels found in the Tables or databases disclosed herein if the expression level varies within a factor of about 2, about 1.5 or about 1.0 fold. In some cases, the expression levels are comparable if the agent induces a change in the expression of a gene in the same direction (e.g., up or down) as a reference toxin.

The cell population that is exposed to the test agent, compound or composition may be exposed in vitro or in vivo. For instance, cultured or freshly isolated hepatocytes, in particular rat hepatocytes, may be exposed to the agent under standard laboratory and cell culture conditions. In another assay format, in vivo exposure may be accomplished by administration of the agent to a living animal, for instance a laboratory rat.

Procedures for designing and conducting toxicity tests in in vitro and in vivo systems are well known, and are described in many texts on the subject, such as Loomis et al., Loomis's Esstentials of Toxicology, 4th Ed., Academic Press, New York, 1996; Echobichon, The Basics of Toxicity Testing, CRC Press, Boca Raton, 1992; Frazier, editor, In Vitro Toxicity Testing, Marcel Dekker, New York, 1992; and the like.

In in vitro toxicity testing, two groups of test organisms are usually employed. One group serves as a control and the other group receives the test compound in a single dose (for acute toxicity tests) or a regimen of doses (for prolonged or chronic toxicity tests). Because, in some cases, the extraction of tissue as called for in the methods of the invention requires sacrificing the test animal, both the control group and the group receiving compound must be large enough to permit removal of animals for sampling tissues, if it is desired to observe the dynamics of gene expression through the duration of an experiment.

In setting up a toxicity study, extensive guidance is provided in the literature for selecting the appropriate test organism for the compound being tested, route of administration. dose ranges, and the like. Water or physiological saline (0.9% NaCl in water) is the solute of choice for the test compound since these solvents permit administration by a variety of routes. When this is not possible because of solubility limitations, vegetable oils such as corn oil or organic solvents such as propylene glycol may be used.

Regardless of the route of administration, the volume required to administer a given dose is limited by the size of the animal that is used. It is desirable to keep the volume of each dose uniform within and between groups of animals. When rats or mice are used, the volume administered by the oral route generally should not exceed about 0.005 ml per gram of animal. Even when aqueous or physiological saline solutions are used for parenteral injection the volumes that are tolerated are limited, although such solutions are ordinarily thought of as being innocuous. The intravenous LD₅₀ of distilled water in the mouse is approximately 0.044 ml per gram and that of isotonic saline is 0.068 ml per gram of mouse. In some instances, the route of administration to the test animal should be the same as, or as similar as possible to, the route of administration of the compound to man for therapeutic purposes.

When a compound is to be administered by inhalation, special techniques for generating test atmospheres are necessary. The methods usually involve aerosolization or nebulization of fluids containing the compound. If the agent to be tested is a fluid that has an appreciable vapor pressure, it may be administered by passing air through the solution under controlled temperature conditions. Under these conditions, dose is estimated from the volume of air inhaled per unit time, the temperature of the solution, and the vapor pressure of the agent involved. Gases are metered from reservoirs. When particles of a solution are to be administered, unless the particle size is less than about 2 μm the particles will not reach the terminal alveolar sacs in the lungs. A variety of apparatuses and chambers are available to perform studies for detecting effects of irritant or other toxic endpoints when they are administered by inhalation. The preferred method of administering an agent to animals is via the oral route, either by intubation or by incorporating the agent in the feed.

When the agent is exposed to cells in vitro or in cell culture, the cell population to be exposed to the agent may be divided into two or more subpopulations, for instance, by dividing the population into two or more identical aliquots. In some preferred embodiments of the methods of the invention, the cells to be exposed to the agent are derived from liver tissue. For instance, cultured or freshly isolated rat hepatocytes may be used.

The methods of the invention may be used to generally predict at least one toxic response, and as described in the Examples, may be used to predict the likelihood that a compound or test agent will induce various specific liver pathologies such as liver necrosis, fatty liver disease, protein adduct formation, hepatitis or other pathologies associated with at least one of the toxins herein described. The methods of the invention may also be used to determine the similarity of a toxic response to one or more individual compounds. In addition, the methods of the invention may be used to predict or elucidate the potential cellular pathways influenced, induced or modulated by the compound or test agent due to the similarity of the expression profile compared to the profile induced by a known toxin (see Tables 5A-5WWW).

Diagnostic Uses for the Toxicity Markers

As described above, the genes and gene expression information or portfolios of the genes with their expression information as provided in Tables 1-5WWW may be used as diagnostic markers for the prediction or identification of the physiological state of tissue or cell sample that has been exposed to a compound or to identify or predict the toxic effects of a compound or agent. For instance, a tissue sample such as a sample of peripheral blood cells or some other easily obtainable tissue sample may be assayed by any of the methods described above, and the expression levels from a gene or genes from Tables 1-5WWW may be compared to the expression levels found in tissues or cells exposed to the toxins described herein. These methods may result in the diagnosis of a physiological state in the cell or may be used to identify the potential toxicity of a compound, for instance a new or unknown compound or agent. The comparison of expression data, as well as available sequence or other information may be done by researcher or diagnostician or may be done with the aid of a computer and databases as described below.

In another format, the levels of a gene(s) of Tables 1-5WWW, its encoded protein(s), or any metabolite produced by the encoded protein may be monitored or detected in a sample, such as a bodily tissue or fluid sample to identify or diagnose a physiological state of an organism. Such samples may include any tissue or fluid sample, including urine, blood and easily obtainable cells such as peripheral lymphocytes.

Use of the Markers for Monitoring Toxicity Progression

As described above, the genes and gene expression information provided in Tables 1-5WWW may also be used as markers for the monitoring of toxicity progression, such as that found after initial exposure to a drug, drug candidate, toxin, pollutant, etc. For instance, a tissue or cell sample may be assayed by any of the methods described above, and the expression levels from a gene or genes from Tables 1-5WWW may be compared to the expression levels found in tissue or cells exposed to the hepatotoxins described herein. The comparison of the expression data, as well as available sequence or other information may be done by researcher or diagnostician or may be done with the aid of a computer and databases.

Use of the Toxicity Markers for Drug Screening

According to the present invention, the genes identified in Tables 1-5WWW may be used as markers or drug targets to evaluate the effects of a candidate drug, chemical compound or other agent on a cell or tissue sample. The genes may also be used as drug targets to screen for agents that modulate their expression and/or activity. In various formats, a candidate drug or agent can be screened for the ability to stimulate the transcription or expression of a given marker or markers or to down-regulate or counteract the transcription or expression of a marker or markers. According to the present invention, one can also compare the specificity of a drug's effects by looking at the number of markers which the drug induces and comparing them. More specific drugs will have less transcriptional targets. Similar sets of markers identified for two drugs may indicate a similarity of effects.

Assays to monitor the expression of a marker or markers as defined in Tables 1-5WWW may utilize any available means of monitoring for changes in the expression level of the nucleic acids of the invention. As used herein, an agent is said to modulate the expression of a nucleic acid of the invention if it is capable of up- or down-regulating expression of the nucleic acid in a cell.

In one assay format, gene chips containing probes to one, two or more genes from Tables 1-5WWW may be used to directly monitor or detect changes in gene expression in the treated or exposed cell. Cell lines, tissues or other samples are first exposed to a test agent and in some instances, a known toxin, and the detected expression levels of one or more, or preferably 2 or more of the genes of Tables 1-5WWW are compared to the expression levels of those same genes exposed to a known toxin alone. Compounds that modulate the expression patterns of the known toxin(s) would be expected to modulate potential toxic physiological effects in vivo. The genes in Tables 1-5WWW are particularly appropriate marks in these assays as they are differentially expressed in cells upon exposure to a known hepatotoxin.

In another format, cell lines that contain reporter gene fusions between the open reading frame and/or the transcriptional regulatory regions of a gene in Tables 1-5WWW and any assayable fusion partner may be prepared. Numerous assayable fusion partners are known and readily available including the firefly luciferase gene and the gene encoding chloramphenicol acetyltransferase (Alam et al. (1990) Anal Biochem 188:245-254). Cell lines containing the reporter gene fusions are then exposed to the agent to be tested under appropriate conditions and time. Differential expression of the reporter gene between samples exposed to the agent and control samples identifies agents which modulate the expression of the nucleic acid.

Additional assay formats may be used to monitor the ability of the agent to modulate the expression of a gene identified in Tables 1-5WWW. For instance, as described above, mRNA expression may be monitored directly by hybridization of probes to the nucleic acids of the invention. Cell lines are exposed to the agent to be tested under appropriate conditions and time and total RNA or mRNA is isolated by standard procedures such those disclosed in Sambrook et al. (Molecular Cloning: A Laboratory Manual, Third Ed., Cold Spring Harbor Laboratory Press, Cold Spring Harbor, N.Y., 2001).

In another assay format, cells or cell lines are first identified which express the gene products of the invention physiologically. Cell and/or cell lines so identified would be expected to comprise the necessary cellular machinery such that the fidelity of modulation of the transcriptional apparatus is maintained with regard to exogenous contact of agent with appropriate surface transduction mechanisms and/or the cytosolic cascades. Further, such cells or cell lines may be transduced or transfected with an expression vehicle (e.g., a plasmid or viral vector) construct comprising an operable non-translated 5′-promoter containing end of the structural gene encoding the gene products of Tables 1-5WWW fused to one or more antigenic fragments or other detectable markers, which are peculiar to the instant gene products, wherein said fragments are under the transcriptional control of said promoter and are expressed as polypeptides whose molecular weight can be distinguished from the naturally occurring polypeptides or may further comprise an immunologically distinct or other detectable tag. Such a process is well known in the art (see Sambrook et al., supra).

Cells or cell lines transduced or transfected as outlined above are then contacted with agents under appropriate conditions; for example, the agent comprises a pharmaceutically acceptable excipient and is contacted with cells comprised in an aqueous physiological buffer such as phosphate buffered saline (PBS) at physiological pH, Eagles balanced salt solution (BSS) at physiological pH, PBS or BSS comprising serum or conditioned media comprising PBS or BSS and/or serum incubated at 37° C. Said conditions may be modulated as deemed necessary by one of skill in the art. Subsequent to contacting the cells with the agent, said cells are disrupted and the polypeptides of the lysate are fractionated such that a polypeptide fraction is pooled and contacted with an antibody to be further processed by immunological assay (e.g., ELISA, immunoprecipitation or Western blot). The pool of proteins isolated from the “agent-contacted” sample is then compared with the control samples (no exposure and exposure to a known toxin) where only the excipient is contacted with the cells and an increase or decrease in the immunologically generated signal from the “agent-contacted” sample compared to the control is used to distinguish the effectiveness and/or toxic effects of the agent.

Another embodiment of the present invention provides methods for identifying agents that modulate at least one activity of a protein(s) encoded by the genes in Tables 1-5WWW. Such methods or assays may utilize any means of monitoring or detecting the desired activity.

In one format, the relative amounts of a protein (Tables 1-5WWW) between a cell population that has been exposed to the agent to be tested compared to an un-exposed control cell population and a cell population exposed to a known toxin may be assayed. In this format, probes such as specific antibodies are used to monitor the differential expression of the protein in the different cell populations. Cell lines or populations are exposed to the agent to be tested under appropriate conditions and time. Cellular lysates may be prepared from the exposed cell line or population and a control, unexposed cell line or population. The cellular lysates are then analyzed with the probe, such as a specific antibody.

Agents that are assayed in the above methods can be randomly selected or rationally selected or designed. As used herein, an agent is said to be randomly selected when the agent is chosen randomly without considering the specific sequences involved in the association of the a protein of the invention alone or with its associated substrates, binding partners, etc. An example of randomly selected agents is the use a chemical library or a peptide combinatorial library, or a growth broth of an organism.

As used herein, an agent is said to be rationally selected or designed when the agent is chosen on a nonrandom basis which takes into account the sequence of the target site and/or its conformation in connection with the agent's action. Agents can be rationally selected or rationally designed by utilizing the peptide sequences that make up these sites. For example, a rationally selected peptide agent can be a peptide whose amino acid sequence is identical to or a derivative of any functional consensus site.

The agents of the present invention can be, as examples, peptides, small molecules, vitamin derivatives, as well as carbohydrates. Dominant negative proteins, DNAs encoding these proteins, antibodies to these proteins, peptide fragments of these proteins or mimics of these proteins may be introduced into cells to affect function. “Mimic” used herein refers to the modification of a region or several regions of a peptide molecule to provide a structure chemically different from the parent peptide but topographically and functionally similar to the parent peptide (see G. A. Grant in: Molecular Biology and Biotechnology, Meyers, ed., pp. 659-664, VCH Publishers, New York, 1995). A skilled artisan can readily recognize that there is no limit as to the structural nature of the agents of the present invention.

Nucleic Acid Assay Formats

The genes identified as being differentially expressed upon exposure to a known hepatotoxin (Tables 1-5WWW) may be used in a variety of nucleic acid detection assays to detect or quantititate the expression level of a gene or multiple genes in a given sample. The genes described in Tables 1-5WWW may also be used in combination with one or more additional genes whose differential expression is associate with toxicity in a cell or tissue. In preferred embodiments, the genes in Tables 1-5WWW may be combined with one or more of the genes described in related U.S. applications 60/222,040, 60/244,880, 60/290,029, 60/290,645, 60/292,336, 60/295,798, 60/297,457, 60/298,884, 60/303,459, 60/331,273, 60/364,045, 60/364,055, 60/436,643, Ser. Nos. 09/917,800 and 10/060,087, all of which are herein incorporated by reference.

Any assay format to detect gene expression may be used. For example, traditional Northern blotting, dot or slot blot, nuclease protection, primer directed amplification, RT-PCR, semi- or quantitative PCR, branched-chain DNA and differential display methods may be used for detecting gene expression levels. Those methods are useful for some embodiments of the invention. In cases where smaller numbers of genes are detected, high throughput amplification-based assays may be most efficient. Methods and assays of the invention, however, may be most efficiently designed with hybridization-based methods for detecting the expression of a large number of genes.

Any hybridization assay format may be used, including solution-based and solid support-based assay formats. Solid supports containing oligonucleotide probes for differentially expressed genes of the invention can be filters, polyvinyl chloride dishes, particles, beads, microparticles or silicon or glass based chips, etc. Such chips, wafers and hybridization methods are widely available, for example, those disclosed by Beattie (WO 95/1755).

Any solid surface to which oligonucleotides can be bound, either directly or indirectly, either covalently or non-covalently, can be used. A preferred solid support is a high density array or DNA chip. These contain a particular oligonucleotide probe in a predetermined location on the array. Each predetermined location may contain more than one molecule of the probe, but each molecule within the predetermined location has an identical sequence. Such predetermined locations are termed features. There may be, for example, from 2, 10, 100, 1000 to 10,000, 100,000 or 400,000 or more of such features on a single solid support. The solid support, or the area within which the probes are attached may be on the order of about a square centimeter. Probes corresponding to the genes of Tables 1-5WWW or from the related applications described above may be attached to single or multiple solid support structures, e.g., the probes may be attached to a single chip or to multiple chips to comprise a chip set.

Oligonucleotide probe arrays for expression monitoring can be made and used according to any techniques known in the art (see for example, Lockhart et al. (1996) Nat Biotechnol 14:1675-1680; McGall et al. (1996) Proc Nat Acad Sci USA 93:13555-13460). Such probe arrays may contain at least two or more oligonucleotides that are complementary to or hybridize to two or more of the genes described in Tables 1-5WWW. For instance, such arrays may contain oligonucleotides that are complementary or hybridize to at least 2, 3, 4, 5, 6, 7, 8, 9, 10, 20, 30, 50, 70, 100 or more the genes described herein. Preferred arrays contain all, substantially all, or nearly all of the genes listed in Tables 1-5WWW, or individually, the gene sets of Tables 5A-5WWW. In another preferred embodiment, arrays are constructed that contain oligonucleotides to detect all or nearly all of the genes in any one of or all of Tables 1-5WWW on a single solid support substrate, such as a chip.

The sequences of the expression marker genes of Tables 1-5WWW are in the public databases. Table 1 provides the GenBank Accession Number for each of the sequences (see www.ncbi.nlm.nih.gov/). The sequences of the genes in GenBank are expressly herein incorporated by reference in their entirety as of the filing date of this application, as are related sequences, for instance, sequences from the same gene of different lengths, variant sequences, polymorphic sequences, genomic sequences of the genes and related sequences from different species, including the human counterparts, where appropriate (see Table 3). These sequences may be used in the methods of the invention or may be used to produce the probes and arrays of the invention. In some embodiments, the genes in Tables 1-5WWW that correspond to the genes or fragments previously associated with a toxic response may be excluded from the Tables.

As described above, in addition to the sequences of the GenBank Accessions Numbers disclosed in the Tables 1-5WWW, sequences such as naturally occurring variant or polymorphic sequences may be used in the methods and compositions of the invention. For instance, expression levels of various allelic or homologous forms of a gene disclosed in the Tables 1-5WWW may be assayed. Any and all nucleotide variations that do not alter the functional activity of a gene listed in the Tables 1-5WWW, including all naturally occurring allelic variants of the genes herein disclosed, may be used in the methods and to make the compositions (e.g., arrays) of the invention.

Probes based on the sequences of the genes described above may be prepared by any commonly available method. Oligonucleotide probes for screening or assaying a tissue or cell sample are preferably of sufficient length to specifically hybridize only to appropriate, complementary genes or transcripts. Typically the oligonucleotide probes will be at least about 10, 12, 14, 16, 18, 20 or 25 nucleotides in length. In some cases, longer probes of at least 30, 40, or 50 nucleotides will be desirable.

As used herein, oligonucleotide sequences that are complementary to one or more of the genes described in Tables 1-5WWW refer to oligonucleotides that are capable of hybridizing under stringent conditions to at least part of the nucleotide sequences of said genes. Such hybridizable oligonucleotides will typically exhibit at least about 75% sequence identity at the nucleotide level to said genes, preferably about 80% or 85% sequence identity or more preferably about 90% or 95% or more sequence identity to said genes.

“Bind(s) substantially” refers to complementary hybridization between a probe nucleic acid and a target nucleic acid and embraces minor mismatches that can be accommodated by reducing the stringency of the hybridization media to achieve the desired detection of the target polynucleotide sequence.

The terms “background” or “background signal intensity” refer to hybridization signals resulting from non-specific binding, or other interactions, between the labeled target nucleic acids and components of the oligonucleotide array (e.g., the oligonucleotide probes, control probes, the array substrate, etc.). Background signals may also be produced by intrinsic fluorescence of the array components themselves. A single background signal can be calculated for the entire array, or a different background signal may be calculated for each target nucleic acid. In a preferred embodiment, background is calculated as the average hybridization signal intensity for the lowest 5% to 10% of the probes in the array, or, where a different background signal is calculated for each target gene, for the lowest 5% to 10% of the probes for each gene. Of course, one of skill in the art will appreciate that where the probes to a particular gene hybridize well and thus appear to be specifically binding to a target sequence, they should not be used in a background signal calculation. Alternatively, background may be calculated as the average hybridization signal intensity produced by hybridization to probes that are not complementary to any sequence found in the sample (e.g. probes directed to nucleic acids of the opposite sense or to genes not found in the sample such as bacterial genes where the sample is mammalian nucleic acids). Background can also be calculated as the average signal intensity produced by regions of the array that lack any probes at all.

The phrase “hybridizing specifically to” refers to the binding, duplexing, or hybridizing of a molecule substantially to or only to a particular nucleotide sequence or sequences under stringent conditions when that sequence is present in a complex mixture (e.g., total cellular) DNA or RNA.

Assays and methods of the invention may utilize available formats to simultaneously screen at least about 100, preferably about 1000, more preferably about 10,000 and most preferably about 100,000 or 1,000,000 or more different nucleic acid hybridizations.

As used herein a “probe” is defined as a nucleic acid, capable of binding to a target nucleic acid of complementary sequence through one or more types of chemical bonds, usually through complementary base pairing, usually through hydrogen bond formation. As used herein, a probe may include natural (i.e., A, G, U, C, or T) or modified bases (7-deazaguanosine, inosine, etc.). In addition, the bases in probes may be joined by a linkage other than a phosphodiester bond, so long as it does not interfere with hybridization. Thus, probes may be peptide nucleic acids in which the constituent bases are joined by peptide bonds rather than phosphodiester linkages.

The term “perfect match probe” refers to a probe that has a sequence that is perfectly complementary to a particular target sequence. The test probe is typically perfectly complementary to a portion (subsequence) of the target sequence. The perfect match (PM) probe can be a “test probe”, a “normalization control” probe, an expression level control probe and the like. A perfect match control or perfect match probe is, however, distinguished from a “mismatch control” or “mismatch probe.”

The terms “mismatch control” or “mismatch probe” refer to a probe whose sequence is deliberately selected not to be perfectly complementary to a particular target sequence. For each mismatch (MM) control in a high-density array there typically exists a corresponding perfect match (PM) probe that is perfectly complementary to the same particular target sequence. The mismatch may comprise one or more bases.

While the mismatch(s) may be located anywhere in the mismatch probe, terminal mismatches are less desirable as a terminal mismatch is less likely to prevent hybridization of the target sequence. In a particularly preferred embodiment, the mismatch is located at or near the center of the probe such that the mismatch is most likely to destabilize the duplex with the target sequence under the test hybridization conditions.

The term “stringent conditions” refers to conditions under which a probe will hybridize to its target subsequence, but with only insubstantial hybridization to other sequences or to other sequences such that the difference may be identified. Stringent conditions are sequence-dependent and will be different in different circumstances. Longer sequences hybridize specifically at higher temperatures. Generally, stringent conditions are selected to be about 5° C. lower than the thermal melting point (Tm) for the specific sequence at a defined ionic strength and pH.

Typically, stringent conditions will be those in which the salt concentration is at least about 0.01 to 1.0 M Na⁺ ion concentration (or other salts) at pH 7.0 to 8.3 and the temperature is at least about 30° C. for short probes (e.g., 10 to 50 nucleotides). Stringent conditions may also be achieved with the addition of destabilizing agents such as formamide.

The “percentage of sequence identity” or “sequence identity” is determined by comparing two optimally aligned sequences or subsequences over a comparison window or span, wherein the portion of the polynucleotide sequence in the comparison window may optionally comprise additions or deletions (i.e., gaps) as compared to the reference sequence (which does not comprise additions or deletions) for optimal alignment of the two sequences. The percentage is calculated by determining the number of positions at which the identical submit (e.g. nucleic acid base or amino acid residue) occurs in both sequences to yield the number of matched positions, dividing the number of matched positions by the total number of positions in the window of comparison and multiplying the result by 100 to yield the percentage of sequence identity. Percentage sequence identity when calculated using the programs GAP or BESTFIT (see below) is calculated using default gap weights.

Probe Design

One of skill in the art will appreciate that an enormous number of array designs are suitable for the practice of this invention. The high density array will typically include a number of test probes that specifically hybridize to the sequences of interest. Probes may be produced from any region of the genes identified in the Tables and the attached representative sequence listing. In instances where the gene reference in the Tables is an EST, probes may be designed from that sequence or from other regions of the corresponding full-length transcript that may be available in any of the sequence databases, such as those herein described. See WO 99/32660 for methods of producing probes for a given gene or genes. In addition, any available software may be used to produce specific probe sequences, including, for instance, software available from Molecular Biology Insights, Olympus Optical Co. and Biosoft International. In a preferred embodiment, the array will also include one or more control probes.

High density array chips of the invention include “test probes.” Test probes may be oligonucleotides that range from about 5 to about 500, or about 7 to about 50 nucleotides, more preferably from about 10 to about 40 nucleotides and most preferably from about 15 to about 35 nucleotides in length. In other particularly preferred embodiments, the probes are about 20 or 25 nucleotides in length. In another preferred embodiment, test probes are double or single strand DNA sequences. DNA sequences are isolated or cloned from natural sources or amplified from natural sources using native nucleic acid as templates. These probes have sequences complementary to particular subsequences of the genes whose expression they are designed to detect. Thus, the test probes are capable of specifically hybridizing to the target nucleic acid they are to detect.

In addition to test probes that bind the target nucleic acid(s) of interest, the high density array can contain a number of control probes. The control probes may fall into three categories referred to herein as 1) normalization controls; 2) expression level controls; and 3) mismatch controls.

Normalization controls are oligonucleotide or other nucleic acid probes that are complementary to labeled reference oligonucleotides or other nucleic acid sequences that are added to the nucleic acid sample to be screened. The signals obtained from the normalization controls after hybridization provide a control for variations in hybridization conditions, label intensity, “reading” efficiency and other factors that may cause the signal of a perfect hybridization to vary between arrays. In a preferred embodiment, signals (e.g., fluorescence intensity) read from all other probes in the array are divided by the signal (e.g., fluorescence intensity) from the control probes thereby normalizing the measurements.

Virtually any probe may serve as a normalization control. However, it is recognized that hybridization efficiency varies with base composition and probe length. Preferred normalization probes are selected to reflect the average length of the other probes present in the array, however, they can be selected to cover a range of lengths. The normalization control(s) can also be selected to reflect the (average) base composition of the other probes in the array, however in a preferred embodiment, only one or a few probes are used and they are selected such that they hybridize well (i.e., no secondary structure) and do not match any target-specific probes.

Expression level controls are probes that hybridize specifically with constitutively expressed genes in the biological sample. Virtually any constitutively expressed gene provides a suitable target for expression level controls. Typically expression level control probes have sequences complementary to subsequences of constitutively expressed “housekeeping genes” including, but not limited to the actin gene, the transferrin receptor gene, the GAPDH gene, and the like.

Mismatch controls may also be provided for the probes to the target genes, for expression level controls or for normalization controls. Mismatch controls are oligonucleotide probes or other nucleic acid probes identical to their corresponding test or control probes except for the presence of one or more mismatched bases. A mismatched base is a base selected so that it is not complementary to the corresponding base in the target sequence to which the probe would otherwise specifically hybridize. One or more mismatches are selected such that under appropriate hybridization conditions (e.g., stringent conditions) the test or control probe would be expected to hybridize with its target sequence, but the mismatch probe would not hybridize (or would hybridize to a significantly lesser extent) Preferred mismatch probes contain a central mismatch. Thus, for example, where a probe is a 20 mer, a corresponding mismatch probe will have the identical sequence except for a single base mismatch (e.g., substituting a G, a C or a T for an A) at any of positions 6 through 14 (the central mismatch).

Mismatch probes thus provide a control for non-specific binding or cross hybridization to a nucleic acid in the sample other than the target to which the probe is directed. For example, if the target is present the perfect match probes should be consistently brighter than the mismatch probes. In addition, if all central mismatches are present, the mismatch probes can be used to detect a mutation, for instance, a mutation of a gene in the accompanying Tables 1-5WWW. The difference in intensity between the perfect match and the mismatch probe provides a good measure of the concentration of the hybridized material.

Nucleic Acid Samples

Cell or tissue samples may be exposed to the test agent in vitro or in vivo. When cultured cells or tissues are used, appropriate mammalian liver extracts may also be added with the test agent to evaluate agents that may require biotransformation to exhibit toxicity. In a preferred format, primary isolates of animal or human hepatocytes which already express the appropriate complement of drug-metabolizing enzymes may be exposed to the test agent without the addition of mammalian liver extracts.

The genes which are assayed according to the present invention are typically in the form of mRNA or reverse transcribed mRNA. The genes may be cloned or not. The genes may be amplified or not. The cloning and/or amplification do not appear to bias the representation of genes within a population. In some assays, it may be preferable, however, to use polyA+ RNA as a source, as it can be used with less processing steps.

As is apparent to one of ordinary skill in the art, nucleic acid samples used in the methods and assays of the invention may be prepared by any available method or process. Methods of isolating total mRNA are well known to those of skill in the art. For example, methods of isolation and purification of nucleic acids are described in detail in Chapter 3 of Laboratory Techniques in Biochemistry and Molecular Biology. Vol. 24, Hybridization With Nucleic Acid Probes: Theory and Nucleic Acid Probes, P. Tijssen, Ed., Elsevier Press, New York, 1993. Such samples include RNA samples, but also include cDNA synthesized from a mRNA sample isolated from a cell or tissue of interest. Such samples also include DNA amplified from the cDNA, and RNA transcribed from the amplified DNA. One of skill in the art would appreciate that it is desirable to inhibit or destroy RNase present in homogenates before homogenates are used.

Biological samples may be of any biological tissue or fluid or cells from any organism as well as cells raised in vitro, such as cell lines and tissue culture cells. Frequently the sample will be a tissue or cell sample that has been exposed to a compound, agent, drug, pharmaceutical composition, potential environmental pollutant or other composition. In some formats, the sample will be a “clinical sample” which is a sample derived from a patient. Typical clinical samples include, but are not limited to, sputum, blood, blood cells (e.g., white cells), tissue or fine needle biopsy samples, urine, peritoneal fluid, and pleural fluid, or cells therefrom.

Biological samples may also include sections of tissues, such as frozen sections or formalin fixed sections taken for histological purposes.

Forming High Density Arrays

Methods of forming high density arrays of oligonucleotides with a minimal number of synthetic steps are known. The oligonucleotide analogue array can be synthesized on a single or on multiple solid substrates by a variety of methods, including, but not limited to, light-directed chemical coupling, and mechanically directed coupling (see Pirrung, U.S. Pat. No. 5,143,854).

In brief, the light-directed combinatorial synthesis of oligonucleotide arrays on a glass surface proceeds using automated phosphoramidite chemistry and chip masking techniques. In one specific implementation, a glass surface is derivatized with a silane reagent containing a functional group, e.g., a hydroxyl or amine group blocked by a photolabile protecting group. Photolysis through a photolithogaphic mask is used selectively to expose functional groups which are then ready to react with incoming 5′ photoprotected nucleoside phosphoramidites. The phosphoramidites react only with those sites which are illuminated (and thus exposed by removal of the photolabile blocking group). Thus, the phosphoramidites only add to those areas selectively exposed from the preceding step. These steps are repeated until the desired array of sequences have been synthesized on the solid surface. Combinatorial synthesis of different oligonucleotide analogues at different locations on the array is determined by the pattern of illumination during synthesis and the order of addition of coupling reagents.

In addition to the foregoing, additional methods which can be used to generate an array of oligonucleotides on a single substrate are described in PCT Publication Nos. WO 93/09668 and WO 01/23614. High density nucleic acid arrays can also be fabricated by depositing pre-made or natural nucleic acids in predetermined positions. Synthesized or natural nucleic acids are deposited on specific locations of a substrate by light directed targeting and oligonucleotide directed targeting. Another embodiment uses a dispenser that moves from region to region to deposit nucleic acids in specific spots.

Hybridization

Nucleic acid hybridization simply involves contacting a probe and target nucleic acid under conditions where the probe and its complementary target can form stable hybrid duplexes through complementary base pairing. See WO 99/32660. The nucleic acids that do not form hybrid duplexes are then washed away leaving the hybridized nucleic acids to be detected, typically through detection of an attached detectable label. It is generally recognized that nucleic acids are denatured by increasing the temperature or decreasing the salt concentration of the buffer containing the nucleic acids. Under low stringency conditions (e.g., low temperature and/or high salt) hybrid duplexes (e.g., DNA:DNA, RNA:RNA, or RNA:DNA) will form even where the annealed sequences are not perfectly complementary. Thus, specificity of hybridization is reduced at lower stringency. Conversely, at higher stringency (e.g., higher temperature or lower salt) successful hybridization tolerates fewer mismatches. One of skill in the art will appreciate that hybridization conditions may be selected to provide any degree of stringency.

In a preferred embodiment, hybridization is performed at low stringency, in this case in 6×SSPET at 37° C. (0.005% Triton X-100), to ensure hybridization and then subsequent washes are performed at higher stringency (e.g., 1×SSPET at 37° C.) to eliminate mismatched hybrid duplexes. Successive washes may be performed at increasingly higher stringency (e.g., down to as low as 0.25×SSPET at 37° C. to 50° C.) until a desired level of hybridization specificity is obtained. Stringency can also be increased by addition of agents such as formamide. Hybridization specificity may be evaluated by comparison of hybridization to the test probes with hybridization to the various controls that can be present (e.g., expression level control, normalization control, mismatch controls, etc.).

In general, there is a tradeoff between hybridization specificity (stringency) and signal intensity. Thus, in a preferred embodiment, the wash is performed at the highest stringency that produces consistent results and that provides a signal intensity greater than approximately 10% of the background intensity. Thus, in a preferred embodiment, the hybridized array may be washed at successively higher stringency solutions and read between each wash. Analysis of the data sets thus produced will reveal a wash stringency above which the hybridization pattern is not appreciably altered and which provides adequate signal for the particular oligonucleotide probes of interest.

Signal Detection

The hybridized nucleic acids are typically detected by detecting one or more labels attached to the sample nucleic acids. The labels may be incorporated by any of a number of means well known to those of skill in the art. See WO 99/32660.

Databases

The present invention includes relational databases containing sequence information, for instance, for the genes of Tables 1-5WWW, as well as gene expression information from tissue or cells exposed to various standard toxins, such as those herein described (see Tables 5A-5WWW). Databases may also contain information associated with a given sequence or tissue sample such as descriptive information about the gene associated with the sequence information (see Table 1), or descriptive information concerning the clinical status of the tissue sample, or the animal from which the sample was derived. The database may be designed to include different parts, for instance a sequence database and a gene expression database. Methods for the configuration and construction of such databases and computer-readable media to which such databases are saved are widely available, for instance, see U.S. Pat. No. 5,953,727, which is herein incorporated by reference in its entirety.

The databases of the invention may be linked to an outside or external database such as GenBank (www.nebi.nlm.nih.gov/entrez.index.html); KEGG (www.genome.ad.jp/kegg); SPAD (www.grt.kyushu-u.ac.jpspad/index.html); HUGO (www.gene.ucl.ac.uk/hugo); Swiss-Prot (www.expasy.ch.sprot); Prosite (www.expasy.ch/tools/scnpsit1.html); OMIM (www.ncbi.nlm.nih.gov/omim); and GDB (www.gdb.org). In a preferred embodiment, as described in Tables 1-3, the external database is GenBank and the associated databases maintained by the National Center for Biotechnology Information (NCBI) (www.ncbi.nlm.nih.gov).

Any appropriate computer platform, user interface, etc. may be used to perform the necessary comparisons between sequence information, gene expression information and any other information in the database or information provided as an input. For example, a large number of computer workstations are available from a variety of manufacturers, such has those available from Silicon Graphics. Client/server environments, database servers and networks are also widely available and appropriate platforms for the databases of the invention.

The databases of the invention may be used to produce, among other things, electronic Northerns that allow the user to determine the cell type or tissue in which a given gene is expressed and to allow determination of the abundance or expression level of a given gene in a particular tissue or cell.

The databases of the invention may also be used to present information identifying the expression level in a tissue or cell of a set of genes comprising one or more of the genes in Tables 1-5WWW, comprising the step of comparing the expression level of at least one gene in Tables 1-5WWW in a cell or tissue exposed to a test agent to the level of expression of the gene in the database. Such methods may be used to predict the toxic potential of a given compound by comparing the level of expression of a gene or genes in Tables 1-5WWW from a tissue or cell sample exposed to the test agent to the expression levels found in a control tissue or cell samples exposed to a standard toxin or hepatotoxin such as those herein described. Such methods may also be used in the drug or agent screening assays as described herein.

Kits

The invention further includes kits combining, in different combinations, high-density oligonucleotide arrays, reagents for use with the arrays, protein reagents encoded by the genes of the Tables, signal detection and array-processing instruments, gene expression databases and analysis and database management software described above. The kits may be used, for example, to predict or model the toxic response of a test compound, to monitor the progression of hepatic disease states, to identify genes that show promise as new drug targets and to screen known and newly designed drugs as discussed above.

The databases packaged with the kits are a compilation of expression patterns from human or laboratory animal genes and gene fragments (corresponding to the genes of Tables 1-5WWW). In particular, the database software and packaged information that may contain the databases saved to a computer-readable medium include the expression results of Tables 1-5WWW that can be used to predict toxicity of a test agent by comparing the expression levels of the genes of Tables 1-5WWW induced by the test agent to the expression levels presented in Tables 1-5WWW. In another format, database and software information may be provided in a remote electronic format, such as a website, the address of which may be packaged in the kit.

The kits may used in the pharmaceutical industry, where the need for early drug testing is strong due to the high costs associated with drug development, but where bioinformatics, in particular gene expression informatics, is still lacking. These kits will reduce the costs, time and risks associated with traditional new drug screening using cell cultures and laboratory animals. The results of large-scale drug screening of pre-grouped patient populations, pharmacogenomics testing, can also be applied to select drugs with greater efficacy and fewer side-effects. The kits may also be used by smaller biotechnology companies and research institutes who do not have the facilities for performing such large-scale testing themselves.

Databases and software designed for use with use with microarrays are discussed in Balaban et al., U.S. Pat. No. 6,229,911, a computer-implemented method for managing information, stored as indexed tables and collected from small or large numbers of microarrays, and U.S. Pat. No. 6,185,561, a computer-based method with data mining capability for collecting gene expression level data, adding additional attributes and reformatting the data to produce answers to various queries. Chee et al., U.S. Pat. No. 5,974,164, discloses a software-based method for identifying mutations in a nucleic acid sequence based on differences in probe fluorescence intensities between wild type and mutant sequences that hybridize to reference sequences.

Without further description, it is believed that one of ordinary skill in the art can, using the preceding description and the following illustrative examples, make and utilize the compounds of the present invention and practice the claimed methods. The following working examples therefore, specifically point out the preferred embodiments of the present invention, and are not to be construed as limiting in any way the remainder of the disclosure.

### EXAMPLES

### Example 1

### Identification of Toxicity Markers

The hepatotoxins 2-acetylaminofluorene (2-AAF), BI liver toxin, chloroform, CI-1000, dimethylnitrosamine (DMN), gemfibrozil, menadione, thioacetamide, acyclovir, AY-25329, bicalutamide, clofibrate, colchicine, diflunisal, dioxin, hydrazine, phenobarbital, valproate, zileuton and LPS were administered to male Sprague-Dawley rats at various time points using administration diluents, protocols and dosing regimes as indicated in Table 6. The hepatotoxins ANIT, acetominophen, carbon tetrachloride, chloroform, CPA, diclofenac, 17α-ethinylestradiol, indomethacin, tacrine and Wy-14643 were administered to male Sprague-Dawley rats at various time points using administration diluents, protocols and dosing regimes as previously described in the art and previously described in the related applications discussed above.

After adminstration, the dosed animals were observed and tissues were collected as described below:

Observation of Animals

1. Clinical Observations—Twice daily: mortality and moribundity check.

Cage Side Observations—skin and fur, eyes and mucous membrane, respiratory system, circulatory system, autonomic and central nervous system, somatomotor pattern, and behavior pattern.

Potential signs of toxicity, including tremors, convulsions, salivation, diarrhea, lethargy, coma or other atypical behavior or appearance, were recorded as they occurred and included a time of onset, degree, and duration.

2. Physical Examinations—Prior to randomization, prior to initial treatment, and prior to sacrifice.

3. Body Weights—Prior to randomization, prior to initial treatment, and prior to sacrifice.

Clinical Pathology

1. Frequency Prior to necropsy.

2. Number of animals All surviving animals.

3. Bleeding Procedure Blood was obtained by puncture of the orbital sinus while under 70% CO₂/30% O₂ anesthesia.

4. Collection of Blood Samples—Approximately 0.5 mL of blood was collected into EDTA tubes for evaluation of hematology parameters. Approximately 1 mL of blood was collected into serum separator tubes for clinical chemistry analysis. Approximately 200 uL of plasma was obtained and frozen at ˜−80° C. for test compound/metabolite estimation. An additional ˜2 mL of blood was collected into a 15 mL conical polypropylene vial to which ˜3 mL of Trizol was immediately added. The contents were immediately mixed with a vortex and by repeated inversion. The tubes were frozen in liquid nitrogen and stored at approximately −80° C.

Termination Procedures

Terminal Sacrifice

At the sampling times indicated in Table 6 for each hepatotoxin, and as previously described in the related applications mentioned above, rats were weighed, physically examined, sacrificed by decapitation, and exsanguinated. The animals were necropsied within approximately five minutes of sacrifice. Separate sterile, disposable instruments were used for each animal, with the exception of bone cutters, which were used to open the skull cap. The bone cutters were dipped in disinfectant solution between animals.

Necropsies were conducted on each animal following procedures approved by board-certified pathologists.

Animals not surviving until terminal sacrifice were discarded without necropsy (following euthanasia by carbon dioxide asphyxiation, if moribund). The approximate time of death for moribund or found dead animals was recorded.

Postmortem Procedures

Fresh and sterile disposable instruments were used to collect tissues. Gloves were worn at all times when handling tissues or vials. All tissues were collected and frozen within approximately 7 minutes of the animal's death. The liver sections were frozen within approximately 2 minutes of the animal's death. The time of euthanasia, an interim time point at freezing of liver sections and kidneys, and time at completion of necropsy were recorded. Tissues were stored at approximately −80° C. or preserved in 10% neutral buffered formalin.

Tissue Collection and Processing

Liver

1. Right medial lobe—snap frozen in liquid nitrogen and stored at ˜−80° C.

2. Left medial lobe—Preserved in 10% neutral-buffered formalin (NBF) and evaluated for gross and microscopic pathology.

3. Left lateral lobe—snap frozen in liquid nitrogen and stored at −80° C.

Heart

A sagittal cross-section containing portions of the two atria and of the two ventricles was preserved in 10% NBF. The remaining heart was frozen in liquid nitrogen and stored at ˜−80° C.

Kidneys (Both)

1. Left—Hemi-dissected; half was preserved in 10% NBF and the remaining half was frozen in liquid nitrogen and stored at ˜−80° C.

2. Right—Hemi-dissected; half was preserved in 10% NBF and the remaining half was frozen in liquid nitrogen and stored at ˜−80° C.

Testes (Both)

A sagittal cross-section of each testis was preserved in 10% NBF. The remaining testes were frozen together in liquid nitrogen and stored at ˜−80° C.

Brain (Whole)

A cross-section of the cerebral hemispheres and of the diencephalon was preserved in 10% NBF, and the rest of the brain was frozen in liquid nitrogen and stored at ˜−80° C.

Bone Marrow

Bone marrow was flushed from each femur using a syringe and fresh, cold RPMI (˜1 mL of RPMI×3 washes per femur) into two separate 15 mL conical vials, labeled to distinguish right from left femur samples. The vials were gently inverted several times after collection and maintained on wet ice.

Microarray sample preparation was conducted with minor modifications, following the protocols set forth in the Affymetrix GeneChip Expression Analysis Manual. Frozen tissue was ground to a powder using a Spex Certiprep 6800 Freezer Mill. Total RNA was extracted with Trizol (GibcoBRL) utilizing the manufacturer's protocol. The total RNA yield for each sample was 200-500 μg per 300 mg tissue weight. mRNA was isolated using the Oligotex mRNA Midi kit (Qiagen) followed by ethanol precipitation. Double stranded cDNA was generated from mRNA using the SuperScript Choice system (GibcoBRL). First strand cDNA synthesis was primed with a T7-(dT24) oligonucleotide. The cDNA was phenol-chloroform extracted and ethanol precipitated to a final concentration of 1 μg/ml. From 2 μg of cDNA, cRNA was synthesized using Ambion's T7 MegaScript in vitro Transcription Kit.

To biotin label the cRNA, nucleotides Bio-11-CTP and Bio-16-UTP (Enzo Diagnostics) were added to the reaction. Following a 37° C. incubation for six hours, impurities were removed from the labeled cRNA following the RNeasy Mini kit protocol (Qiagen). cRNA was fragmented (fragmentation buffer consisting of 200 mM Tris-acetate, pH 8.1, 500 mM KOAc, 150 mM MgOAc) for thirty-five minutes at 94° C. Following the Affymetrix protocol, 55 μg of fragmented cRNA was hybridized on the Affymetrix rat array set for twenty-four hours at 60 rpm in a 45° C. hybridization oven. The chips were washed and stained with Streptavidin Phycoerythrin (SAPE) (Molecular Probes) in Affymetrix fluidics stations. To amplify staining, SAPE solution was added twice with an anti-streptavidin biotinylated antibody (Vector Laboratories) staining step in between. Hybridization to the probe arrays was detected by fluorometric scanning (Hewlett Packard Gene Array Scanner). Data was analyzed using Affymetrix GeneChip® version 3.0 and Expression Data Mining (EDMT) software (version 1.0), GeneExpress2000, and S-Plus.

Table 1 discloses a set of genes that are differentially expressed upon exposure to the named toxins and their corresponding GenBank Accession and Sequence Identification numbers, the gene names if known, and the sequence cluster titles (core set and alternate set gene fragments). The human homologues of the rat genes in Table 1 are indicated in Table 3. The identities of the metabolic pathways in which the genes of Table 1 function are indicated in Table 2. The model codes in Tables 1-3 represent the various toxicity or liver pathology states that differential expression of each gene is able to identify, as well as the individual toxin or toxin type associated with differential expression of each gene. The model codes are defined in Table 4. The GLGC ID is the internal Gene Logic identification number.

Tables 5A-5WWW disclose a core or alternate set of genes, along with the summary statistics for each of the comparisons performed as indicated in these tables—i.e., expression levels of a particular gene in toxicity group samples compared to non-toxicity group samples in response to exposure to a particular toxin, or as measured in a particular disease state. Each of these tables contains a set of predictive genes and creates a model for predicting the hepatoxicity of an unknown, i.e., untested compound. Each gene is identified by its Gene Logic identification number and can be cross-referenced to a gene name and representative SEQ ID NO. in Table 1 or in one more related applications, as mentioned on page 1. For each comparison of gene expression levels between samples in the toxicity group (samples affected by exposure to a toxin) and samples in the non-toxicity group (samples not affected by exposure to a toxin), the tox mean (for toxicity group samples) is the mean signal intensity, as normalized for the various chip parameters that are being assayed. The non-tox mean represents the mean signal intensity, as normalized for the various chip parameters that are being assayed, in non-toxicity group samples. For individual genes, an increase in the tox mean compared to the non-tox mean indicates up-regulation upon exposure to a toxin, while a decrease in the group mean compared to the non-group mean indicates down-regulation.

The mean values are derived from Average Difference (AveDiff) values for a particular gene, averaged across the corresponding samples. Each individual Average Difference value is calculated by integrating the intensity information from multiple probe pairs that are tiled for a particular fragment. The normalization multiplies each expression intensity for a given experiment (chip) by a global scaling factor. The intent of this normalization is to make comparisons of individual genes between chips possible. The scaling factor is calculated as follows:
1. From all the unnormalized expression values in the experiment, delete the largest 2% and smallest 2% of the values. That is, if the experiment yields 10,000 expression values, order the values and delete the smallest 200 and largest 200.
2. Compute the trimmed mean, which is equal to the mean of the remaining values.
3. Compute the scale factor SF=100/(trimmed mean)

The value of 100 used here is the standard target valued used.

Values greater than 2.0*SD noise are assumed to come from expressors. For these values, the standard deviation SD log (signal) of the logarithms is calculated. The logarithms are then multiplied by a scale factor proportional to 1/SD log (signal) and exponentiated. The resulting values are then multiplied by another scale factor, chosen so there will be no discontinuity in the normalized values from unscaled values on either side of 2.0*SD noise. Some AveDiff values may be negative due to the general noise involved in nucleic acid hybridization experiments. Although many conclusions can be made corresponding to a negative value on the GeneChip platform, it is difficult to assess the meaning behind the negative value for individual fragments. Our observations show that, though negative values are observed at times within the predictive gene set, these values reflect a real biological phenomenon that is highly reproducible across all the samples from which the measurement was taken. For this reason, those genes that exhibit a negative value are included in the predictive set. It should be noted that other platforms of gene expression measurement may be able to resolve the negative numbers for the corresponding genes. The predictive ability of each of those genes should extend across platforms, however. Each mean value is accompanied by the standard deviation for the mean. The linear discriminant analysis score (discriminant score, or LDA), as disclosed in the tables, measures the ability of each gene to predict whether or not a sample is toxic. The discriminant score is calculated by the following steps:

Calculation of a Discriminant Score

Let Xᵢ represent the AveDiff values for a given gene across the Group 1 samples, i=1 . . . n.

Let Yᵢ represent the AveDiff values for a given gene across the Group 2 samples, i=1 . . . t.

The calculations proceed as follows:

Calculate mean and standard deviation for Xᵢ's and Yᵢ's, and denote these by m_{X}, m_{Y}, s_{X},s_{Y}.

For all Xᵢ's and Yᵢ's, evaluate the function f(z)=((1/s_{Y})*exp(−0.5*((z−m_{Y}/s_{Y})²))/(((1/s_{Y})*exp(−0.5*((z−m_{Y})/s_{Y})²))+((1/s_{X})*exp(−0.5*((z−m_{X})/s_{X})²))).

The number of correct predictions, say P, is then the number of Yᵢ's such that f(Yᵢ)>0.5 plus the number of Xᵢ's such that f(Xᵢ)<0.5.

The discriminant score is then P/(n+t).

Linear discriminant analysis (LDA) uses both the individual measurements of each gene and the calculated measurements of all combinations of genes to classify samples. For each gene a weight is derived from the mean and standard deviation of the tox and nontox groups. Every gene is multiplied by a weight and the sum of these values results in a collective discriminate score. This discriminant score is then compared against collective centroids of the tox and nontox groups. These centroids are the average of all tox and nontox samples respectively. Therefore, each gene contributes to the overall prediction. This contribution is dependent on weights that are large positive or negative numbers if the relative distances between the tox and nontox samples for that gene are large and small numbers if the relative distances are small. The discriminant score for each unknown sample and centroid values can be used to calculate a probability between zero and one as to the group in which the unknown sample belongs.

### Example 2

### General Toxicity Modeling

Samples were selected for grouping into tox-responding and non-tox-responding groups by examining each study individually with Principal Components Analysis (PCA) to determine which treatments had an observable response. Only groups where confidence of their tox-responding and non-tox-responding status was established were included in building a general tox model.

Linear discriminant models were generated to describe toxic and non-toxic samples. The top discriminant genes and/or EST's were used to determine toxicity by calculating each gene's contribution with homo and heteroscedastic treatment of variance and inclusion or exclusion of mutual information between genes. Prediction of samples within the database exceeded 80% true positives with a false positive rate of less than 5%. It was determined that combinations of genes and/or EST's generally provided a better predictive ability than individual genes and that the more genes and/or EST used the better predictive ability. Although the preferred embodiment includes fifty or more genes, many pairings or greater combinations of genes and/or EST can work better than individual genes. All combinations of two or more genes from the selected list could be used to predict toxicity. These combinations could be selected by pairing in an agglomerate, divisive, or random approach. Further, as yet undetermined genes and/or EST's could be combined with individual or combination of genes and/or EST's described here to increase predictive ability. However, the genes and/or EST's described here would contribute most of the predictive ability of any such undetermined combinations.

Other variations on the above method can provide adequate predictive ability. These include selective inclusion of components via agglomerate, divisive, or random approaches or extraction of loading and combining them in agglomerate, divisive, or random approaches. Also the use of composite variables in logistic regression to determine classification of samples can also be accomplished with linear discriminate analysis, neural or Bayesian networks, or other forms of regression and classification based on categorical or continual dependent and independent variables.

### Example 3

### Modeling with Core Gene Set

As described in Examples 1 and 2, above, the data collected from microarray hybridization experiments were analyzed by LDA and by PCA. The genes in Tables 5A, 5C, 5D, 5E, 5F, 5G, 5I, 5K, 5L, 5M, 5N, 5O, 5Q, 5S, 5T, 5U, 5V, 5W, 5X, 5Z, 5BB, 5DD, 5FF, 5GG, 5HH, 5II, 5JJ, 5LL, 5MM, 5NN, 5PP, 5RR, 5SS, 5TT, 5UU, 5VV, 5WW, 5XX, 5ZZ, 5BBB, 5DDD, 5EEE, 5FFF, 5GGG, 5HHH, 5III, 5KKK, 5LLL, 5MMM, 5NNN, 5OOO, 5PPP, 5RRR, 5SSS, 5TTT, 5UUU and 5VVV constitute a core set of markers for predicting the hepatotoxicity of a compound. The genes in Tables 5B, 5H, 5J, 5P, 5R, 5Y, 5AA, 5CC, 5EE, 5KK, 5OO, 5QQ, 5YY, 5AAA, 5CCC, 5JJJ, 5QQQ, and 5WWW constitute an alternate set of markers which may also be used in the methods of the invention, although the core marker sets of Tables 5A, 5C, 5D, 5E, 5F, 5G, 5I, 5K, 5L, 5M, 5N, 5O, 5Q, 5S, 5T, 5U, 5V, 5W, 5X, 5Z, 5BB, 5DD, 5FF, 5GG, 5HH, 5II, 5JJ, 5LL, 5MM, 5NN, 5PP, 5RR, 5SS, 5TT, 5UU, 5VV, 5WW, 5XX, 5ZZ, 5BBB, 5DDD, 5EEE, 5FFF, 5GGG, 5HHH, 5III, 5KKK, 5LLL, 5MMM, 5NNN, 5OOO, 5PPP, 5RRR, 5SSS, 5TTT, 5UUU and 5VVV may be preferred in some embodiments of the invention because the core sets contain additional predictive genes. Each gene fragment in Tables 1-5WWW is assigned an LDA score, and those gene fragments in the core set are those with the highest LDA scores. The gene fragments in Tables 5A-5WWW were determined to give greater than 80% true positive results and less than 5% false positive results. Gene expression profiles prepared from expression data for these genes, in the presence and absence of toxin treatment, can be used a controls in assays of compounds whose toxic properties have not been examined. Comparison of data from test compound-exposed and test compound-unexposed animals with the data in Tables 5A-5WWW, or with data from the core gene set controls, allows the prediction of toxic effects—or no toxic effects—upon exposure to the test compound. Thus, with a smaller gene set than in Table 1 and as described in Example 1, the core gene set can be used to examine the biological effects of a compound whose toxic properties following exposure are not known and to predict the toxicity in liver tissue of this compound.

### Example 4

### Modeling Methods

The above modeling methods provide broad approaches of combining the expression of genes to predict sample toxicity. One method uses each variable individually and weights them; the other combines variables as a composite measure and adds weights to them after combination into a new variable. One could also provide no weight in a simple voting method or determine weights in a supervised or unsupervised method using agglomerate, divisive, or random approaches. All or selected combinations of genes may be combined in ordered, agglomerate, or divisive, supervised or unsupervised clustering algorithms with unknown samples for classification. Any form of correlation matrix may also be used to classify unknown samples. The spread of the group distribution and discriminate score alone provide enough information to enable a skilled person to generate all of the above types of models with accuracy that can exceed discriminate ability of individual genes. Some examples of methods that could be used individually or in combination after transformation of data types include but are not limited to: Discriminant Analysis, Multiple Discriminant Analysis, logistic regression, multiple regression analysis, linear regression analysis, conjoint analysis, canonical correlation, hierarchical cluster analysis, k-means cluster analysis, self-organizing maps, multidimensional scaling, structural equation modeling, support vector machine determined boundaries, factor analysis, neural networks, bayesian classifications, and resampling methods.

### Example 5

### Grouping of Individual compound and Pathology Classes

Samples were grouped into individual pathology classes based on known toxicological responses and observed clinical chemical and pathology measurements or into early and late phases of observable toxicity within a compound (Tables 1-5WWW). The top 10, 25, 50, 100 genes based on individual discriminate scores were used in a model to ensure that combination of genes provided a better prediction than individual genes. As described above, all combinations of two or more genes from this list could potentially provide better prediction than individual genes when selected in any order or by ordered, agglomerate, divisive, or random approaches. In addition, combining these genes with other genes could provide better predictive ability, but most of this predictive ability would come from the genes listed herein.

Samples may be considered toxic if they score positive in any pathological or individual compound class represented here or in any modeling method mentioned under general toxicology models based on combination of individual time and dose grouping of individual toxic compounds obtainable from the data. The pathological groupings and early and late phase models are preferred examples of all obtainable combinations of sample time and dose points. Most logical groupings with one or more genes and one or more sample dose and time points should produce better predictions of general toxicity, pathological specific toxicity, or similarity to known toxicant than individual genes.

Although the present invention has been described in detail with reference to examples above, it is understood that various modifications can be made without departing from the spirit of the invention. Accordingly, the invention is limited only by the following claims. All cited patents, patent applications and publications referred to in this application are herein incorporated by reference in their entirety.

LENGTHY TABLE REFERENCED HERE
US20070027634A1-20070201-T00001
Please refer to the end of the specification for access instructions.

LENGTHY TABLE REFERENCED HERE
US20070027634A1-20070201-T00002
Please refer to the end of the specification for access instructions.

LENGTHY TABLE REFERENCED HERE
US20070027634A1-20070201-T00003
Please refer to the end of the specification for access instructions.

LENGTHY TABLE REFERENCED HERE
US20070027634A1-20070201-T00004
Please refer to the end of the specification for access instructions.

LENGTHY TABLE REFERENCED HERE
US20070027634A1-20070201-T00005
Please refer to the end of the specification for access instructions.

LENGTHY TABLE REFERENCED HERE
US20070027634A1-20070201-T00006
Please refer to the end of the specification for access instructions.

LENGTHY TABLE REFERENCED HERE
US20070027634A1-20070201-T00007
Please refer to the end of the specification for access instructions.

LENGTHY TABLE REFERENCED HERE
US20070027634A1-20070201-T00008
Please refer to the end of the specification for access instructions.

LENGTHY TABLE REFERENCED HERE
US20070027634A1-20070201-T00009
Please refer to the end of the specification for access instructions.

LENGTHY TABLE REFERENCED HERE
US20070027634A1-20070201-T00010
Please refer to the end of the specification for access instructions.

LENGTHY TABLE REFERENCED HERE
US20070027634A1-20070201-T00011
Please refer to the end of the specification for access instructions.

LENGTHY TABLE REFERENCED HERE
US20070027634A1-20070201-T00012
Please refer to the end of the specification for access instructions.

LENGTHY TABLE REFERENCED HERE
US20070027634A1-20070201-T00013
Please refer to the end of the specification for access instructions.

LENGTHY TABLE REFERENCED HERE
US20070027634A1-20070201-T00014
Please refer to the end of the specification for access instructions.

LENGTHY TABLE REFERENCED HERE
US20070027634A1-20070201-T00015
Please refer to the end of the specification for access instructions.

LENGTHY TABLE REFERENCED HERE
US20070027634A1-20070201-T00016
Please refer to the end of the specification for access instructions.

LENGTHY TABLE REFERENCED HERE
US20070027634A1-20070201-T00017
Please refer to the end of the specification for access instructions.

LENGTHY TABLE REFERENCED HERE
US20070027634A1-20070201-T00018
Please refer to the end of the specification for access instructions.

LENGTHY TABLE REFERENCED HERE
US20070027634A1-20070201-T00019
Please refer to the end of the specification for access instructions.

LENGTHY TABLE REFERENCED HERE
US20070027634A1-20070201-T00020
Please refer to the end of the specification for access instructions.

LENGTHY TABLE REFERENCED HERE
US20070027634A1-20070201-T00021
Please refer to the end of the specification for access instructions.

LENGTHY TABLE REFERENCED HERE
US20070027634A1-20070201-T00022
Please refer to the end of the specification for access instructions.

LENGTHY TABLE REFERENCED HERE
US20070027634A1-20070201-T00023
Please refer to the end of the specification for access instructions.

LENGTHY TABLE REFERENCED HERE
US20070027634A1-20070201-T00024
Please refer to the end of the specification for access instructions.

LENGTHY TABLE REFERENCED HERE
US20070027634A1-20070201-T00025
Please refer to the end of the specification for access instructions.

LENGTHY TABLE REFERENCED HERE
US20070027634A1-20070201-T00026
Please refer to the end of the specification for access instructions.

LENGTHY TABLE REFERENCED HERE
US20070027634A1-20070201-T00027
Please refer to the end of the specification for access instructions.

LENGTHY TABLE REFERENCED HERE
US20070027634A1-20070201-T00028
Please refer to the end of the specification for access instructions.

LENGTHY TABLE REFERENCED HERE
US20070027634A1-20070201-T00029
Please refer to the end of the specification for access instructions.

LENGTHY TABLE REFERENCED HERE
US20070027634A1-20070201-T00030
Please refer to the end of the specification for access instructions.

LENGTHY TABLE REFERENCED HERE
US20070027634A1-20070201-T00031
Please refer to the end of the specification for access instructions.

LENGTHY TABLE REFERENCED HERE
US20070027634A1-20070201-T00032
Please refer to the end of the specification for access instructions.

LENGTHY TABLE REFERENCED HERE
US20070027634A1-20070201-T00033
Please refer to the end of the specification for access instructions.

LENGTHY TABLE REFERENCED HERE
US20070027634A1-20070201-T00034
Please refer to the end of the specification for access instructions.

LENGTHY TABLE REFERENCED HERE
US20070027634A1-20070201-T00035
Please refer to the end of the specification for access instructions.

LENGTHY TABLE REFERENCED HERE
US20070027634A1-20070201-T00036
Please refer to the end of the specification for access instructions.

LENGTHY TABLE REFERENCED HERE
US20070027634A1-20070201-T00037
Please refer to the end of the specification for access instructions.

LENGTHY TABLE REFERENCED HERE
US20070027634A1-20070201-T00038
Please refer to the end of the specification for access instructions.

LENGTHY TABLE REFERENCED HERE
US20070027634A1-20070201-T00039
Please refer to the end of the specification for access instructions.

LENGTHY TABLE REFERENCED HERE
US20070027634A1-20070201-T00040
Please refer to the end of the specification for access instructions.

LENGTHY TABLE REFERENCED HERE
US20070027634A1-20070201-T00041
Please refer to the end of the specification for access instructions.

LENGTHY TABLE REFERENCED HERE
US20070027634A1-20070201-T00042
Please refer to the end of the specification for access instructions.

LENGTHY TABLE REFERENCED HERE
US20070027634A1-20070201-T00043
Please refer to the end of the specification for access instructions.

LENGTHY TABLE REFERENCED HERE
US20070027634A1-20070201-T00044
Please refer to the end of the specification for access instructions.

LENGTHY TABLE REFERENCED HERE
US20070027634A1-20070201-T00045
Please refer to the end of the specification for access instructions.

LENGTHY TABLE REFERENCED HERE
US20070027634A1-20070201-T00046
Please refer to the end of the specification for access instructions.

LENGTHY TABLE REFERENCED HERE
US20070027634A1-20070201-T00047
Please refer to the end of the specification for access instructions.

LENGTHY TABLE REFERENCED HERE
US20070027634A1-20070201-T00048
Please refer to the end of the specification for access instructions.

LENGTHY TABLE REFERENCED HERE
US20070027634A1-20070201-T00049
Please refer to the end of the specification for access instructions.

LENGTHY TABLE REFERENCED HERE
US20070027634A1-20070201-T00050
Please refer to the end of the specification for access instructions.

LENGTHY TABLE REFERENCED HERE
US20070027634A1-20070201-T00051
Please refer to the end of the specification for access instructions.

LENGTHY TABLE REFERENCED HERE
US20070027634A1-20070201-T00052
Please refer to the end of the specification for access instructions.

LENGTHY TABLE REFERENCED HERE
US20070027634A1-20070201-T00053
Please refer to the end of the specification for access instructions.

LENGTHY TABLE REFERENCED HERE
US20070027634A1-20070201-T00054
Please refer to the end of the specification for access instructions.

LENGTHY TABLE REFERENCED HERE
US20070027634A1-20070201-T00055
Please refer to the end of the specification for access instructions.

LENGTHY TABLE REFERENCED HERE
US20070027634A1-20070201-T00056
Please refer to the end of the specification for access instructions.

LENGTHY TABLE REFERENCED HERE
US20070027634A1-20070201-T00057
Please refer to the end of the specification for access instructions.

LENGTHY TABLE REFERENCED HERE
US20070027634A1-20070201-T00058
Please refer to the end of the specification for access instructions.

LENGTHY TABLE REFERENCED HERE
US20070027634A1-20070201-T00059
Please refer to the end of the specification for access instructions.

LENGTHY TABLE REFERENCED HERE
US20070027634A1-20070201-T00060
Please refer to the end of the specification for access instructions.

LENGTHY TABLE REFERENCED HERE
US20070027634A1-20070201-T00061
Please refer to the end of the specification for access instructions.

LENGTHY TABLE REFERENCED HERE
US20070027634A1-20070201-T00062
Please refer to the end of the specification for access instructions.

LENGTHY TABLE REFERENCED HERE
US20070027634A1-20070201-T00063
Please refer to the end of the specification for access instructions.

LENGTHY TABLE REFERENCED HERE
US20070027634A1-20070201-T00064
Please refer to the end of the specification for access instructions.

LENGTHY TABLE REFERENCED HERE
US20070027634A1-20070201-T00065
Please refer to the end of the specification for access instructions.

LENGTHY TABLE REFERENCED HERE
US20070027634A1-20070201-T00066
Please refer to the end of the specification for access instructions.

LENGTHY TABLE REFERENCED HERE
US20070027634A1-20070201-T00067
Please refer to the end of the specification for access instructions.

LENGTHY TABLE REFERENCED HERE
US20070027634A1-20070201-T00068
Please refer to the end of the specification for access instructions.

LENGTHY TABLE REFERENCED HERE
US20070027634A1-20070201-T00069
Please refer to the end of the specification for access instructions.

LENGTHY TABLE REFERENCED HERE
US20070027634A1-20070201-T00070
Please refer to the end of the specification for access instructions.

LENGTHY TABLE REFERENCED HERE
US20070027634A1-20070201-T00071
Please refer to the end of the specification for access instructions.

LENGTHY TABLE REFERENCED HERE
US20070027634A1-20070201-T00072
Please refer to the end of the specification for access instructions.

LENGTHY TABLE REFERENCED HERE
US20070027634A1-20070201-T00073
Please refer to the end of the specification for access instructions.

LENGTHY TABLE REFERENCED HERE
US20070027634A1-20070201-T00074
Please refer to the end of the specification for access instructions.

LENGTHY TABLE REFERENCED HERE
US20070027634A1-20070201-T00075
Please refer to the end of the specification for access instructions.

LENGTHY TABLE REFERENCED HERE
US20070027634A1-20070201-T00076
Please refer to the end of the specification for access instructions.

LENGTHY TABLE REFERENCED HERE
US20070027634A1-20070201-T00077
Please refer to the end of the specification for access instructions.

LENGTHY TABLE REFERENCED HERE
US20070027634A1-20070201-T00078
Please refer to the end of the specification for access instructions.

LENGTHY TABLE REFERENCED HERE
US20070027634A1-20070201-T00079
Please refer to the end of the specification for access instructions.

LENGTHY TABLE REFERENCED HERE
US20070027634A1-20070201-T00080
Please refer to the end of the specification for access instructions.

LENGTHY TABLE REFERENCED HERE
US20070027634A1-20070201-T00081
Please refer to the end of the specification for access instructions.

LENGTHY TABLE
The patent application contains a lengthy table section. A copy of the table is available in electronic form from the USPTO web site (http://seqdata.uspto.gov/?pageRequest=docDetail&DocID=US20070027634A1) An electronic copy of the table will also be available from the USPTO upon request and payment of the fee set forth in 37 CFR 1.19(b)(3).

1. A method of predicting at least one toxic effect of a compound, comprising:
(a) preparing a gene expression profile of a tissue or cell sample exposed to the compound; and
(b) comparing the gene expression profile to a database comprising at least part of the data or information of Tables 1-5.

2. A method of claim 1, wherein the gene expression profile prepared from the tissue or cell sample comprises the level of expression for at least one gene.

3. A method of claim 2, wherein the level of expression is compared to a Tox Mean and/or Non-Tox Mean value in Tables 1-5.

4. A method of claim 3, wherein the level of expression is normalized prior to comparison.

5. A method of claim 4, wherein the database comprises substantially all of the data or information in Tables 1-5.

6. A method of claim 1, wherein the tissue or cell sample is a liver tissue or liver cell sample.

7. A method of predicting at least one toxic effect of a compound, comprising:
(a) detecting the level of expression in a tissue or cell sample exposed to the compound of two or more genes from Tables 5B, 5H, 5J, 5P, 5R, 5Y, 5AA, 5CC, 5EE, 5KK, 5OO, 5QQ, 5YY, 5AAA, 5CCC, 5JJJ, 5QQQ, and 5WWW; wherein differential expression of the genes in Tables 5B, 5H, 5J, 5P, 5R, 5Y, 5AA, 5CC, 5EE, 5KK, 5OO, 5QQ, 5YY, 5AAA, 5CCC, 5JJJ, 5QQQ, and 5WWW is indicative of at least one toxic effect.

8. A method of predicting the progression of a toxic effect of a compound, comprising:
(a) detecting the level of expression in a tissue or cell sample exposed to the compound of two or more genes from Tables 5B, 5H, 5J, 5P, 5R, 5Y, 5AA, 5CC, 5EE, 5KK, 5OO, 5QQ, 5YY, 5AAA, 5CCC, 5JJJ, 5QQQ, and 5WWW, wherein differential expression of the genes in Tables 5B, 5H, 5J, 5P, 5R, 5Y, 5AA, 5CC, 5EE, 5KK, 5OO, 5QQ, 5YY, 5AAA, 5CCC, 5JJJ, 5QQQ, and 5WWW is indicative of toxicity progression.

9. A method of predicting the hepatotoxicity of a compound, comprising:
(a) detecting the level of expression in a tissue or cell sample exposed to the compound of two or more genes from Tables 5B, 5H, 5J, 5P, 5R, 5Y, 5AA, 5CC, 5EE, 5KK, 5OO, 5QQ, 5YY, 5AAA, 5CCC, 5JJJ, 5QQQ, and 5WWW, wherein differential expression of the genes in Tables 5B, 5H, 5J, 5P, 5R, 5Y, 5AA, 5CC, 5EE, 5KK, 5OO, 5QQ, 5YY, 5AAA, 5CCC, 5JJJ, 5QQQ, and 5WWW is indicative of hepatotoxicity.

10. A method of identifying an agent that modulates the onset or progression of a toxic response, comprising:
(a) exposing a cell to the agent and a known toxin; and
(b) detecting the expression level of two or more genes from Tables 5B, 5H, 5J, 5P, 5R, 5Y, 5AA, 5CC, 5EE, 5KK, 5OO, 5QQ, 5YY, 5AAA, 5CCC, 5JJJ, 5QQQ, and 5WWW; wherein differential expression of the genes in Tables 1-3 is indicative of toxicity.

11. A method of predicting the cellular pathways that a compound modulates in a cell, comprising:
(a) detecting the level of expression in a tissue or cell sample exposed to the compound of two or more genes from Tables 5B, 5H, 5J, 5P, 5R, 5Y, 5AA, 5CC, 5EE, 5KK, 5OO, 5QQ, 5YY, 5AAA, 5CCC, 5JJJ, 5QQQ, and 5WWW, wherein differential expression of the genes in Tables 5B, 5H, 5J, 5P, 5R, 5Y, 5AA, 5CC, 5EE, 5KK, 5OO, 5QQ, 5YY, 5AAA, 5CCC, 5JJJ, 5QQQ, and 5WWW is associated the modulation of at least one cellular pathway.

12. The method of claim 7, wherein the expression levels of at least 3 genes are detected.

13. The method of claim 7, wherein the expression levels of at least 4 genes are detected.

14. The method of claim 7, wherein the expression levels of at least 5 genes are detected.

15. The method of claim 7, wherein the expression levels of at least 6 genes are detected.

16. The method of claim 7, wherein the expression levels of at least 7 genes are detected.

17. The method of claim 7, wherein the expression levels of at least 8 genes are detected.

18. The method of claim 1, wherein the expression levels of at least 9 genes are detected.

19. The method of claim 1, wherein the expression levels of at least 10 genes are detected.

20. A method of claim 7, wherein the effect is selected from the group consisting of carcinogenesis, cholestasis, hepatitis, liver enlargement, inflammation, liver necrosis, liver steatosis and peroxisome proliferation.

21. A method of claim 9, wherein the hepatotoxicity is associated with at least one liver disease pathology selected from the group consisting of carcinogenesis, cholestasis, hepatitis, liver enlargement, inflammation, liver necrosis, liver steatosis and peroxisome proliferation.

22. A method of claim 11, wherein the cellular pathway is modulated by a toxin selected from the group consisting of acetominophen, 2-acetylaminofluorene (2-AAF), acyclovir, ANIT, AY-25329, BI liver toxin, chloroform, bicalutamide, carbon tetrachloride, chloroform, CI-1000, clofibrate, colchicine, CPA, diclofenac, diflunisal, dimethylnitrosamine (DMN), dioxin, 17α-ethinylestradiol, gemfibrozil, hydrazine, indomethacin, LPS, menadione, phenobarbital, tacrine, thioacetamide, valproate, Wy-14643 and zileuton.

23. A set of at least two probes, wherein each of the probes comprises a sequence that specifically hybridizes to a gene in Tables 5B, 5H, 5J, 5P, 5R, 5Y, 5AA, 5CC, 5EE, 5KK, 5OO, 5QQ, 5YY, 5AAA, 5CCC, 5JJJ, 5QQQ, and 5WWW.

24. A set of probes according to claim 23, wherein the set comprises probes that hybridize to at least 3 genes.

25. A set of probes according to claim 23, wherein the set comprises probes that hybridize to at least 5 genes.

26. A set of probes according to claim 23, wherein the set comprises probes that hybridize to at least 7 genes.

27. A set of probes according to claim 23, wherein the set comprises probes that hybridize to at least 10 genes.

28. A set of probes according to claim 23, wherein the probes are attached to a solid support.

29. A set of probes according to claim 28, wherein the solid support is selected from the group consisting of a membrane, a glass support and a silicon support.

30. A solid support comprising at least two probes, wherein each of the probes comprises a sequence that specifically hybridizes to a gene in Tables 5B, 5H, 5J, 5P, 5R, 5Y, 5AA, 5CC, 5EE, 5KK, 5OO, 5QQ, 5YY, 5AAA, 5CCC, 5JJJ, 5QQQ, and 5WWW.

31. A solid support of claim 30, wherein the solid support is an array comprising at least 10 different oligonucleotides in discrete locations per square centimeter.

32. A solid support of claim 31, wherein the array comprises at least about 100 different oligonucleotides in discrete locations per square centimeter.

33. A solid support of claim 31, wherein the array comprises at least about 1000 different oligonucleotides in discrete locations per square centimeter.

34. A solid support of claim 31, wherein the array comprises at least about 10,000 different oligonucleotides in discrete locations per square centimeter.

35. A computer system comprising:
(a) a database containing information identifying the expression level in a tissue or cell sample exposed to a hepatotoxin of a set of genes comprising at least two genes in Tables 5B, 5H, 5J, 5P, 5R, 5Y, 5AA, 5CC, 5EE, 5KK, 5OO, 5QQ, 5YY, 5AAA, 5CCC, 5JJJ, 5QQQ, and 5WWW; and
(b) a user interface to view the information.

36. A computer system of claim 35, wherein the database further comprises sequence information for the genes.

37. A computer system of claim 35, wherein the database further comprises information identifying the expression level for the set of genes in the tissue or cell sample before exposure to a hepatotoxin.

38. A computer system of claim 35, wherein the database further comprises information identifying the expression level of the set of genes in a tissue or cell sample exposed to at least a second hepatotoxin.

39. A computer system of claim 35, further comprising records including descriptive information from an external database, which information correlates said genes to records in the external database.

40. A computer system of claim 39, wherein the external database is GenBank.

41. A method of using a computer system of claim 35 to present information identifying the expression level in a tissue or cell of at least one gene in Tables 5B, 5H, 5J, 5P, 5R, 5Y, 5AA, 5CC, 5EE, 5KK, 5OO, 5QQ, 5YY, 5AAA, 5CCC, 5JJJ, 5QQQ, and 5WWW, comprising:
(a) comparing the expression level of at least one gene in Tables 1-3 in a tissue or cell exposed to a test agent to the level of expression of the gene in the database.

42. A method of claim 41, wherein the expression levels of at least two genes are compared.

43. A method of claim 41, wherein the expression levels of at least five genes are compared.

44. A method of claim 41, wherein the expression levels of at least ten genes are compared.

45. A method of claim 41, further comprising the step of displaying the level of expression of at least one gene in the tissue or cell sample compared to the expression level when exposed to a toxin.

46. A method of claim 10, wherein the known toxin is a hepatotoxin.

47. A method of claim 43, wherein the hepatotoxin is selected from the group consisting of acetominophen, 2-acetylaminofluorene (2-AAF), acyclovir, ANIT, AY-25329, BI liver toxin, chloroform, bicalutamide, carbon tetrachloride, chloroform, CI-1000, clofibrate, colchicine, CPA, diclofenac, diflunisal, dimethylnitrosamine (DMN), dioxin, 170α-ethinylestradiol, gemfibrozil, hydrazine, indomethacin, LPS, menadione, phenobarbital, tacrine, thioacetamide, valproate, Wy-14643 and zileuton.

48. A method of claim 7, wherein nearly all of the genes in Tables 5B, 5H, 5J, 5P, 5R, 5Y, 5AA, 5CC, 5EE, 5KK, 5OO, 5QQ, 5YY, 5AAA, 5CCC, 5JJJ, 5QQQ, and 5WWW are detected.

49. A method of claim 48, wherein all of the genes in at least one of Tables 5B, 5H, 5J, 5P, 5R, 5Y, 5AA, 5CC, 5EE, 5KK, 5OO, 5QQ, 5YY, 5AAA, 5CCC, 5JJJ, 5QQQ, and 5WWW are detected.

50. A kit comprising at least one solid support of claim 30 packaged with gene expression information for said genes.

51. A kit of claim 50, wherein the gene expression information comprises gene expression levels in a tissue or cell sample exposed to a hepatotoxin.

52. A kit of claim 51, wherein the gene expression information is in an electronic format.

53. A method of claim 7, wherein the compound exposure is in vivo or in vitro.

54. A method of claim 7, wherein the level of expression is detected by an amplification or hybridization assay.

55. A method of claim 54, wherein the amplification assay is quantitative or semi-quantitative PCR.

56. A method of claim 54, wherein the hybridization assay is selected from the group consisting of Northern blot, dot or slot blot, nuclease protection and microarray assays.

57. A method of identifying an agent that modulates at least one activity of a protein encoded by a gene in Tables 5B, 5H, 5J, 5P, 5R, 5Y, 5AA, 5CC, 5EE, 5KK, 5OO, 5QQ, 5YY, 5AAA, 5CCC, 5JJJ, 5QQQ, and 5WWW comprising:
(a) exposing the protein to the agent; and
(b) assaying at least one activity of said protein.

58. A method of claim 57, wherein the agent is exposed to a cell expressing the protein.

59. A method of claim 58, wherein the cell is exposed to a known toxin.

60. A method of claim 59, wherein the toxin modulates the expression of the protein.

61. A method of claim 1, wherein the level of expression is compared to a Tox Mean and/or Non-Tox Mean value in Tables 5A-5WWW.

62. A method of claim 61, wherein the level of expression is normalized prior to comparison.

63. A method of claim 62, wherein the tissue or cell sample is a liver tissue or liver cell sample.

64. A computer system comprising:
(a) a database containing information identifying the expression level in a tissue or cell sample exposed to a hepatotoxin of a set of genes comprising substantially all of the genes in Tables 5A, 5C, 5D, 5E, 5F, 5G, 5I, 5K, 5L, 5M, 5N, 5O, 5Q, 5S, 5T, 5U, 5V, 5W, 5X, 5Z, 5BB, 5DD, 5FF, 5GG, 5HH, 5II, 5JJ, 5LL, 5MM, 5NN, 5PP, 5RR, 5SS, 5TT, 5UU, 5VV, 5WW, 5XX, 5ZZ, 5BBB, 5DDD, 5EEE, 5FFF, 5GGG, 5HHH, 5III, 5KKK, 5LLL, 5MMM, 5NNN, 5OOO, 5PPP, 5RRR, 5SSS, 5TTT, 5UUU and 5VVV; and
(b) a user interface to view the information.

65. An array comprising probes which individually specifically hybridize to substantially all of the genes in Tables 5A, 5C, 5D, 5E, 5F, 5G, 5I, 5K, 5L, 5M, 5N, 5O, 5Q, 5S, 5T, 5U, 5V, 5W, 5X, 5Z, 5BB, 5DD, 5FF, 5GG, 5HH, 5II, 5JJ, 5LL, 5MM, 5NN, 5PP, 5RR, 5SS, 5TT, 5UU, 5VV, 5WW, 5XX, 5ZZ, 5BBB, 5DDD, 5EEE, 5FFF, 5GGG, 5HHH, 5III, 5KKK, 5LLL, 5MMM, 5NNN, 5OOO, 5PPP, 5RRR, 5SSS, 5TTT, 5UUU and 5VVV.
